(19) United States
(12) Patent Application Publication
(10) Pub. No.: US 2023/0258643 A1
(43) Pub. Date: Aug. 17, 2023

(54) ENDOGENOUS RETROVIRUSES FOR THE DETECTION, DIAGNOSIS, AND PROGNOSIS OF CANCER

(71) Applicant: SunnyBay Bio Tech, Inc., Fremont, CA (US)

(72) Inventors: Gary JOHANNING, Sunnyvale, CA (US); Feng WANG-JOHANNING, Sunnyvale, CA (US)

(73) Assignee: SunnyBay Bio Tech, Inc., Fremont, CA (US)

(21) Appl. No.: 17/995,855

(22) PCT Filed: Apr. 9, 2021
(86) PCT No.: PCT/IB2021/000288

§ 371 Date: Oct. 8, 2022

Related U.S. Application Data
(60) Provisional application No. 63/007,784, filed on Apr. 9, 2020.

(51) Int. Cl. G01N 33/574; C12Q 1/6886; C12Q 1/70

(52) U.S. Cl. CPC G01N 33/57484; C12Q 1/6886; C12Q 1/702; C12Q 2600/156; C12Q 2600/158; G01N 2333/15

(57) ABSTRACT

The invention provides for the early detection, diagnostic, and prognostic assays and assay kits for breast cancer and other cancers, based upon the detection of human endogenous retroviruses (HERVs) as cancer biomarkers.

### REFERENCE TO RELATED APPLICATIONS

This patent application is a National Stage under 35 U.S.C. § 371 of International Application No. PCT/IB2021/000288, filed Apr. 9, 2021, which claims the benefit of U.S. Provisional Patent Application No. 63/007,784, filed Apr. 9, 2020. The disclosures of these applications are incorporated by reference herein in their entirety.

### FIELD OF THE INVENTION

This invention generally relates to immunoassays and bispecific binding assays; materials therefor for cancer involving compounds serving as markers for tumor, cancer, neoplasia, and specifically relates to tumor-associated antigen immunological test systems.

### SEQUENCE LISTING

The instant application contains a Sequence Listing which has been submitted electronically in ASCII format and is hereby incorporated by reference in its entirety. Said ASCII copy, created on Apr. 4, 2023, is named 124712.US003_ST25.txt and is 4,096 bytes in size.

### BACKGROUND OF THE INVENTION

Human endogenous retroviruses (HERVs) are remnants of ancient germ cell 25 infections carried in the genome. Millions of years ago, HERVs infected our human ancestors and integrated themselves into the human genome. HERV genes have mutated into non-infective and essentially un-expressed “sleeper genes.”

HERVs are generally noninfectious, but a few HERV types including human endogenous retrovirus type K (HERV-K or HERV-K(HML-2)) contain open reading frames 30 and are transcriptionally active in human cancer tissues. Most human cancer cells reactivate the expression of latent HERV proteins. This reactivation occurs at early stages of breast cancer, which make HERVs excellent biomarkers of early breast cancer.

An important consideration when developing a new cancer biomarker is the expression profile of the tumor associated antigen. HERV-K is transcriptionally active in 35 germ cell tumors, melanoma, breast cancer cell lines, breast cancer tissues, and ovarian cancer. The inventors specifically identified HERV proteins and sequences in cancer cell lines, patient tumors and blood samples. The inventors observed the expression of HERVs, especially HERV-K sequences, in breast, lung, prostate, ovarian, colon, pancreatic, and other solid tumors. They also found that the expression of HERV-K env transcripts in breast cancer was specifically associated with the progression of neoplasia, such that later stage tumors increase the expression of HERV-K.

There remains a need in the oncological art for biomarkers of early cancers.

### SUMMARY OF THE INVENTION

The invention provides for early cancer detection and for diagnostic, and prognostic assays and assay kits for breast cancer and other cancers, based upon the detection of human endogenous retroviruses (HERVs) as cancer biomarkers. In one aspect, the invention provides the discovery of a pan-cancer HERV biomarker does not appear in individuals without cancer. When HERV is over-expressed in a human cancer, this over-expression generates a cancer-specific gene and antigen expression and a cancer-specific antibody (Ab) response. The inventors have identified that cancer is correlated with HERV expression and HERV antibody response.

In another aspect, the invention provides the discovery that the immune system sees HERV as foreign and generates antibodies against it (autoantibodies), which can be detected, using the invention's compositions and methods, in the subject's tissue, including in serum.

In a first embodiment, the invention provides articles of manufacture, which are assay kits for the analysis of biomarkers. The invention provides a kit for detecting HERV antigen or a cell expressing HERV in a sample, such as in bodily fluids. The kit comprises an anti-HERV antibody of the invention. The anti-HERV antibody can be conjugated to a detection agent or contrast agent. The assay kit optionally also contains instructions for the kit. These assay kits can advantageously be used for treating, detecting, or diagnosing HERV-mediated diseases or disorders. In a second embodiment, the invention provides assay kits for the analysis of human endogenous retroviruses (HERVs) as cancer biomarkers. These assay kits are based on the discovery that blood antibodies against an immunogenic HERV antigen can serve as a biomarker. An advantage of the articles of manufacture is early detection of cancer, such as ductal carcinoma in situ (DCIS).

In a third embodiment, the invention provides an assay kit to directly assay bodily fluids for the presence of HERV antigen. The kit contains one or more reagents sufficient for detection of the presence or absence of an anti-HERV antibody or an HERV target in a sample. The sample assayed for the presence of a HERV antigen in a subject is not limited. The sample can be tissue, e.g., a breast biopsy sample or a biopsy sample from another normal or diseased human organ. The sample can be blood, serum, plasma, urine, circulating cells, e.g., peripheral blood mononuclear cells (PBMCs), urine, saliva, or semen, or a component thereof.

In a fourth embodiment, the invention provides anti-HERV antibodies for use in a lateral flow immunoassay with a strip similar to those in pregnancy test kits. Advantageously, the assay can be done on-site in a medical clinic. This HERV antigen detector device comprises a test strip where the detection reagent is anti-HERV antibody, such as mAb 6H5 conjugated to horseradish peroxidase, where the detectably labeled detection reagent specifically binds HERV antigen, and where the binding of HERV antigen to the detection reagent allows detection of the level of HERV antigen in the sample in the form of colorimetric indicators for reporting whether the target molecule is present. The visual colorimetric indicators correlate the presence of cancer. The lateral flow immunoassay test strip is then visualized to ascertain a positive screen for cancer.

In a fifth embodiment, the invention provides an assay kit for the detection of a HERV antibody. The sample assayed for the presence of an anti-HERV antibody in a subject is not limited. The sample can be tissue, e.g., a breast biopsy sample or a biopsy sample from another normal or diseased human organ. The sample can be blood, serum, plasma, urine, circulating cells, e.g., peripheral blood mononuclear cells (PBMCs), urine, saliva, or semen, or a component thereof. The assay kits are useful for detecting antibodies in the blood that specifically bind to one or more of the antigen, the epitope, and the derivative. Antibodies against HERV were found to be more prominent in patients with carcinomas than with other diseases. The human immune system thus sees the invention's HERV antigens as foreign and generates an antibody response against it. Individuals with inappropriately elevated levels of antibodies against the invention's HERV antigens thus have an increased risk of cancer because the immune system is detecting the presence of the autoantigen in occult cancerous cells. Changes in the levels of these antibodies can screen for the development or progression of cancer. Data show that antibodies against HERV surface (SU) and transmembrane (TM) proteins have the promising ability to distinguish patients with carcinomas compared to healthy controls. Such autoantibodies have potential for diagnostic and prognostic approaches to human cancers. These autoantibodies against HERV show improved sensitivity and specificity for cancer versus normal serum, as compared to prior art cancer markers. Thus, the invention arises from the discovery that measuring an increase in the level of these can be used for the early detection of a variety of cancers, particularly carcinomas.

In a sixth embodiment, the invention provides an assay kit for the detection of a HERV target in a sample. This invention is based on the discovery that blood HERV nucleic acids that include RNA and DNA can serve as a biomarker. The sample assayed for the presence of an HERV target in a subject is not limited. The sample can be tissue, e.g., a breast biopsy sample or a biopsy sample from another normal or diseased human organ. The sample can be blood, serum, plasma, urine, circulating cells, e.g., peripheral blood mononuclear cells (PBMCs), urine, saliva, or semen, or a component thereof. HERV RNA are useful biomarkers for detection and monitoring of cancer, including early detection of carcinomas.

In a seventh embodiment, the invention provides an assay kit for the detection of several markers indicating the presence of HERV in a sample. These articles of manufacture are based on markers expressed in both normal and breast cancer sera. These HERV-K assays measure a single protein expressed at high levels in sera of patients with early to late stage breast cancer but not in normal sera. This strategy targets endogenous viral antigens found only on cancer cells—not on normal tissues. the inventors have generated monoclonal antibodies that bind selectively to these viral antigens including the HERV-K envelope (Env) [surface (SU) and transmembrane (TM)], Gag, NP9, and Rec proteins, and that can detect cancer at early stages and throughout the stages of cancer progression. These non-human proteins can be exploited as ideal targets for cancer early detection, diagnosis, and prognosis, and as companion diagnostics for therapeutic antibodies and other constructs that target HERV-K.

In an eighth embodiment, the invention provides an assay system determine the activity of cancer cells toward combinations of agents or a single agent selectively targeting HERV. The system comprises a preparation of cancer cells; combinations of agents or a single agent selectively targeting HERV; and one or more reagents sufficient to perform an assay selected from groups that comprise (1) cell growth or survival assays carried out under specific culture conditions, (2) the ability to express a defined biologic factor, (3) cell structure assays, or (4) differential gene expression assays. Kit components include primers, buffers, probes, primary antibodies, and secondary antibodies for immunolabeling and signal detection to increase signal amplification and sensitivity. Secondary antibodies can be conjugated to enzymes such as horseradish peroxidase (HRP) or alkaline phosphatase (AP); or fluorescent dyes such as fluorescein isothiocyanate (FITC), rhodamine derivatives, Alexa Fluor dyes, or other molecules to be used in various applications, enzymes, e.g., polymerases, ligases, reverse transcriptase's, nucleases, components for sample isolation, sample preparation, instrumentation, software, instructions for cancer detection, diagnosis, and prognosis in the subject based on the presence or absence of targets that comprise HERV antigens, HERV RNA, or anti-HERV antibodies. The instructions provide recommendations to assist a treating physician in the course of action, based on the results of the analysis, to optimize patient care.

In an ninth embodiment, the invention provides an anti-HERV antibody. In a tenth embodiment, the anti-HERV antibody is mAb 6H5.

In an eleventh embodiment, the invention provides nucleotides, such as primers, for detecting nucleic acids (RNA or DNA) that correspond to the viral genome or subtypes of the viral genome.

In a twelfth embodiment, the invention provides biomarker panels comprising endogenous retroviruses and immune checkpoint proteins. These biomarkers and biomarker panels are useful in the early detection, diagnosis, and prognosis of cancers, such as epithelial cancers. The biomarkers and biomarker panels are exemplified by (a) HERV antigens, (b) epitopes of the HERV antigens, (c) derivatives of the HERV antigen, (d) blood antibodies that specifically bind to one or more of the antigen, the epitope, and the derivative, combinations of HERV antigen or anti-HERV antibodies and immune checkpoint proteins (ICPs), and (e) HERV viral reverse transcriptase, RNA or DNA circulating in the blood. In a thirteenth embodiment, the use of the biomarker panels is a multiplexing approach to analyze candidate tumor associated HERV antigens, and immune checkpoint proteins in blood and other bodily fluids, to identify a biomarker combination with the highest power to detect early stage cancer. The set of serum endogenous retroviral markers includes serum tumor associated HERV antigens and serum antibodies targeting HERV. The results provided by the assay kits include detecting in a sample from the subject an increased level, relative to a control sample, of one or more HERV antigens, epitopes of HERV antigen, and derivatives of the HERV antigen, wherein the HERV antigen is selected from but not limited to the group of full length HERV, full length HERV-K, HERV Env protein [surface (SU) and transmembrane (TM)], HERV-K Env protein [surface (SU) and transmembrane (TM)], HERV Gag, Pol, NP9, and Rec proteins, and HERV-K Gag, Pol, NP9, and Rec proteins. the invention provides compositions and methods for detecting cancer in a subject, comprising detecting in a sample from the subject an increased level, relative to a control sample, of one or more anti-HERV-K antibodies targeting HERV-K antigens, one or more anti-HERV antibodies targeting HERV antigens, epitopes of HERV or HERV-K antigen, and derivatives of the HERV or HERV-K antigen, wherein the HERV or HERV-K antigen is selected from, but not limited to, the group of full length HERV, full length HERV-K, full length HERV Env protein [surface (SU) and transmembrane (TM)], HERV-K Env protein [surface (SU) and transmembrane (TM)], Gag, Pol, NP9, and Rec proteins.

In a fourteenth embodiment, a panel of biomarkers for cancer early detection, diagnosis, and prognosis comprises bodily fluid HERV levels and immune checkpoint proteins, bodily fluid anti-HERV antibody titers and immune checkpoint proteins, and bodily fluid HERV RNA levels and immune checkpoint proteins. The HERV/ICP-associated signature was an unknown common matrix response to human cancers and demonstrates the biomarkers and biomarker panels of the invention are of prognostic and diagnostic significance for a range of cancers. The biomarkers and biomarker panels also present a potential for targeting treatment to a consistent feature of many cancers. The invention thus provides use of the biomarker panels of the invention or subset selection thereof in a method of detection, diagnosis, and prognosis of cancer. Such uses are generally in vitro or ex vivo uses. The invention also provides the use of the biomarker panels of the invention, or subset selection thereof, in manufacturing a biosensor, such as a microarray, or an ELISA assay or a quantitative reverse transcriptase-polymerase chain reaction (qRT-PCR) assay, suitable for detection or quantification of each of the biomarkers. The ELISA, qRT-PCR, and RT-PCR assays provide detection, diagnosis, and prognosis of a HERV-mediated disease.

In an fifteenth embodiment, the invention provides methods of treatment, methods of detection, methods of diagnosis, and methods of prognosis of disease, e.g., the disease of cancer, in a subject using the biomarkers and biomarker panels of the invention. The steps of the methods comprise detecting changes in a set of serum endogenous retroviral markers correlating with the disease, followed by other steps as appropriate for the methods of treatment, methods of detection, methods of diagnosis, and methods of prognosis of disease.

In a sixteenth embodiment, the invention provides a method of cancer detection, diagnosis, and prognosis in a subject comprising: contacting of a sample from a subject with one or more reagents sufficient for detecting an HERV antigen or an anti-HERV blood antibody; measuring an amount of the HERV antigen or anti-HERV blood antibody in the sample; and detecting cancer, the risk of cancer, or the risk of cancer metastasis in the subject based on the amount of the HERV antigen or anti-HERV blood antibody in the sample. The subject is a human subject. The cancer is selected from a group comprising breast cancer, prostate cancer, pancreatic cancer, ovarian cancer, colon cancer, lung cancer, and melanoma. The said sample is selected from a group consisting of a blood or blood derivative sample, a plasma sample, a serum sample, a tissue biopsy, an effusion, a tissue or organ to be transplanted, or a blood product to be transfused. In a seventeenth embodiment, the invention provides methods for detecting cancer or the risk of cancer in a subject. In an eighteenth embodiment, the invention provides that the detecting is a decrease of anti-HERV antibodies or HERV DNA or RNA copies/mL after therapy. In a nineteenth embodiment, the invention's compositions and methods can be applied to a subject that lacks detectable symptoms of cancer, useful in, for example, early screening, a subject at risk of cancer, useful in, for example, early screening, and a subject with one or more detectable symptoms of cancer, useful in, for example, confirmation of a diagnosis of cancer. Thus, the invention's compositions and methods can monitor the progress of cancer or cancer treatment.

In a twentieth embodiment, the invention provides methods for the early detection, diagnosis, and prognosis of cancer, for prediction of response to cancer therapy, and for predicting cancer metastasis.

In a twenty-first embodiment, the invention provides methods for evaluating cancer risk in a subject, comprising: obtaining a blood or other bodily fluid sample from a subject selected for evaluation based on a determination that the subject is at risk of cancer; performing one or more assays configured to detect anti-HERV antibodies by introducing the sample obtained from the subject into an assay instrument which (i) contacts the sample with one or more HERV antigens which specifically bind for detection of the anti-HERV antibody biomarker which are assayed, and (ii) generates one or more assay results indicative of binding of each biomarker which is assayed to a respective HERV antigen to provide one or more assay results; and correlating the assay results generated by the assay instrument to the cancer status of the subject, wherein the correlation step comprises correlating the assay results to one or more of risk stratification, staging, prognosis, classifying and monitoring of the cancer status of the subject, wherein said correlating step comprises assigning a likelihood of one or more future changes in cancer status to the subject based on the assay results.

In a twenty-second embodiment, the invention provides methods for assaying anti-HERV antibodies in a body sample from a subject that can be used for early cancer detection and in diagnostic testing.

In a twenty-third embodiment, the invention provides a method for measurement of reverse transcriptase enzyme activity of a functional and active reverse transcriptase enzyme of HERV in a subject, and detecting cancer, the risk of cancer, or the risk of cancer metastasis in the subject based on the amount of the HERV reverse transcriptase activity in the sample.

In a twenty-fourth embodiment, the invention provides methods for detecting a response to cancer therapy in a subject. In a twenty-fifth embodiment, the invention provides the detection of a response to cancer therapy comprises detecting a response to therapy for cancer metastasis in a subject. In a twenty-sixth embodiment, the invention provides that the detecting is a decrease of anti-HERV antibodies or HERV RNA copies/mL after therapy.

In a twenty-seventh embodiment, the invention provides methods for screening compounds for efficacy. The methods comprise the steps of (a) providing samples from subjects with a cancer; (b) providing reagents sufficient for the detection of an anti-HERV antibody or an HERV target; (b) providing one or more assay compounds for contacting the biological sample; and (d) detecting an amount of the anti-HERV antibody or HERV target in the sample using the reagents. In a twenty-eighth embodiment, the sample is an in vitro sample. In a twenty-ninth embodiment, the sample is an in vivo sample.

### BRIEF DESCRIPTION OF THE DRAWINGS

For illustration, some embodiments of the invention are shown in the drawings described below. Like numerals in the drawings indicate like elements throughout. The invention is not limited to the precise arrangements, dimensions, and instruments shown.

FIG. 1 is a pair of scatter-plot charts showing the results of enzyme-linked immunosorbent assays (ELISA) for anti-HERV-K transmembrane (TM) antibody levels in serum from human breast cancer patients (FIG. 1(A)) and human ovarian cancer patients (FIG. 1(B)). The inventors carried out ELISA assays to detect anti-HERV-K transmembrane antibody levels in serum at a dilution of 1:100 from patients with breast cancer at different stages or from healthy donors. Purified HERV-K transmembrane fusion protein was the antigen. Horizontal bars show the mean. Each point represents an average of three measurements for each sample. The significant difference among the groups was analyzed by ANOVA. FIG. 1 shows that the tendency for the transmembrane antibody level to be increased was likely related to the severity of breast cancer. TIS: Neoplasm of breast primary tumor staging category Tis (DCIS): Ductal carcinoma in situ.

FIG. 2 is a scatter-plot chart showing the results of ELISA assays for anti-HERV-K surface (SU) antibody levels and HERV-K splice variant Np9 and Rec antibody levels in serum from human breast cancer and melanoma patients. The inventors carried out ELISA assays to detect anti-HERV-K surface antibody level in serum at a dilution of 1:100 from patients with breast cancer at different stages or from healthy donors. Purified HERV-K surface-fusion protein was the antigen. Horizontal bars are shown as the mean and each point represents an average of three measurements for each sample. The significant difference among the groups was analyzed by ANOVA. FIG. 2 shows that the tendency for the anti-surface antibody level to be increased was likely related to the severity of breast cancer. TIS: Neoplasm of breast primary tumor staging category Tis (DCIS): Ductal carcinoma in situ.

FIG. 3 shows the detection of anti-HERV antigen antibodies in melanoma patient sera. FIG. 3(A) shows the results of assays where serial dilutions of patient sera were ELISA-assayed for antibody activity against HERV K, Np9, and Rec recombinant proteins. FIG. 3(A) shows that an initial screen of sera from patients with different cancer types found that melanoma patients have enhanced antibody reactivity against HERV antigens, especially Np9 and Rec.

FIG. 4 is a scatter-plot chart showing antibody titers in ELISA assays of sera from patients with TIS [Neoplasm of breast primary tumor staging category Tis (DCIS): Ductal carcinoma in situ] or control subjects without cancer, using a HERV-K MAP peptide as antigen coated on the ELISA plate.

FIG. 5 provides three sets of results. FIG. 5(A) shows the comparison of expression of six immune checkpoint proteins (ICPs) in ductal carcinoma in situ, aggressive breast cancer (aBC), and normal female donors. FIG. 5(B)(a-c) shows the expression of immune checkpoint proteins was decreased post-surgery (6 months; Timepoint 2). FIG. 5(C)(a-d) shows in higher immune checkpoint protein levels were observed in breast cancer patients with higher titers of anti-HERV-K antibody.

FIG. 6 is a set of graphs showing examples of individual fraction reverse transcriptase activity results. Peaks were assayed by RT-PCR using HERV-K10 and HERV-K22 primers.

FIG. 7 is a set dot plots of (A) invasive breast cancer patient, (B) in situ breast cancer patient, (C) malignant ovarian disease patient, and (D) ovarian benign disease patient plasma fraction reverse transcriptase assay results by pool.

FIG. 8 is a set dot plots. FIG. 8(A) is dot plot of patient plasma reverse transcriptase assay pool value t-test for DCIS patient vs. normal donor samples. dA-dD are DCIS patient pools and nA-nD are normal patient pools. FIG. 8(B) is a dot plot of patient plasma reverse transcriptase assay pool value t-test for invasive breast cancer patient vs. normal donor samples. iA-iD are invasive breast cancer patient pools and nA-nD are normal donor pools. FIG. 8(C) patient plasma reverse transcriptase assay pool value t-test for malignant and benign ovarian cancer patients compared to normal donor samples. oA-oD are ovarian cancer plasma pools, bA-bD are benign tumor patient pools, and nA-nD are normal donor plasma pools. Stars indicate p<0.05 comparing the groups indicated.

FIG. 9 is a set of plots. FIG. 9(A) shows the fraction density, Western blot results, and reverse transcriptase activity of Patient Acc. #136. FIG. 9(B) shows that reverse transcriptase activity was compared in cell culture media from various breast cell lines. Serial dilutions of murine leukemia virus reverse transcriptase (Stratagene, La Jolla, Calif., USA) were used as calibrators. Greater reverse transcriptase activity (P<0.001) was demonstrated in all BC cell lines than in MCF-10A or MCF-10AT cells using one-way ANOVA assay. FIG. 9(C) is a scatter plot showing RT-PCR, which was used to verify the expression of HERV-K in sera. Increased expression of HERV-K viral mRNA was observed in BC patient samples with accession (Acc.) #155 and Acc. #73. No expression of HERV-K viral mRNAs was detected in a patient sample (Acc. #163), who had a low HERV-K copy number by quantitative reverse transcriptase-polymerase chain reaction (qRT-PCR). FIG. 9(D) is a scatter plot showing viral load (HERV-K mRNA copy number), which was determined in RNA obtained from patient sera by qRT-PCR. A higher viral load was found in all breast cancer sera, especially in ductal carcinoma in situ DCIS, and in all melanoma sera, compared with normal donors.

FIG. 10 is a drawing showing the structure of a lateral flow immunoassay to detect anti-HERV-K antibodies in the sera of breast cancer patients.

FIG. 11 is a drawing showing a summary of indirect ELISA assay to detect anti-HERV-K serum antibodies.

FIG. 12 is a line graph showing the quantitative measurement of anti-HERV-K IgG concentration. A standard curve of the relationship between optical density and the concentration of an internal standard control IgG mAb, anti-HERV-K mAb 6H5.

FIG. 13 is a graph showing the results of an ELISA assay to evaluate potential high-dose hook effect.

### DETAILED DESCRIPTION OF THE INVENTION

Some aspects, modes, embodiments, variations and features of the invention are described below in various levels of detail to provide a substantial understanding of the invention.

### Industrial Applicability

Breast cancer is the most common malignancy in American women, with approximately 266,120 new cases diagnosed in 2018. Breast cancer is the second leading cause of death by cancer among women.

Several diagnostic products are used for breast cancer disease monitoring or as companion diagnostics for patient selection. The Oncotype DX assay is based on a multigene expression profile of messenger RNAs for 250 candidate genes implicated in breast cancer pathogenesis. The PAM50 breast cancer classifier algorithm uses a microarray to classify patients into subtypes based on the expression patterns of fifty genes. The MammaPrint assay evaluates a 70-gene signature predictive of the early development of distant metastasis. EndoPredict assays eleven genes to predict prognosis of distant recurrence in patients with ER-positive, HER2-negative breast cancer.

The ideal tumor target is broadly expressed on many solid tumors, but it is not expressed on normal adult tissues. The ancient retrovirus HERV-K has this ideal expression profile. The ancient retrovirus HERV-K is highly expressed in many solid tumors but not in normal tissues, suggesting a strong potential as a universal cancer biomarker. This is a different approach than is usually attempted by those having ordinary skill in the oncological art.

The most biologically active HERVs are members of the HERV-K family. HERV-K has a complete sequence capable of expressing all the elements needed for a replication-competent retrovirus but has remained silent in normal cells. The inventors and others reported that, sometimes, such as in tumors, expression of HERV-K is activated and its envelope (Env) protein can be detected in several different types of tumor at much higher levels than in normal tissues. This indicates that HERV-K could be an excellent tumor associated antigen and an ideal target for cancer immunotherapy, because it is expressed in tumors and is absent in normal tissues, which would minimize off-target effects.

Unlike the detection of prostate specific antigen (PSA) this invention arises from the cancer specificity by HERV nucleic acid production (RNA and DNA) or the human antibody response against the invention's HERV antigen. By assaying antibodies against the invention's HERV the inventors can amplify the sensitivity for detection of the invention's HERV antigen. Therapeutic methods to reduce cancer risk by altering these antibody levels can be monitored and used to adjust treatment. Thus, the invention can be used in the prediction of increased risk of developing cancer, early diagnosis of cancer, risk of cancer metastasis, and pharmacodynamic monitoring of immune or other treatments for patients at increased risk of developing cancer due to anti-HERV antibodies.

The invention's article of manufacture, compositions and methods can be used with any cancer, such as carcinoma. The cancer can be selected by medical personnel from a group that includes breast cancer, prostate cancer, ovarian cancer, lung cancer, colon cancer, pancreatic cancer, and melanoma. the cancer is selected from the group of metastatic cancer, e.g., metastatic carcinoma, and non-metastatic cancer, e.g., non-metastatic carcinoma.

### Article of Manufacture

In a thirtieth embodiment, the article of manufacture is the HERV-K ANTIBODY ELISA is a kit designed, with other clinical and diagnostic procedures, to monitor breast cancer recurrence or response to therapy. The medical device can be used for breast cancer patients.

The HERV-K ANTIBODY ELISA includes reagents for detection of circulating serum antibodies against HERV-K in these patients. The kit can be assembled by persons having ordinary skill in the biotechnological art for use in a clinical laboratory. Sera to be assayed can be derived from women with female breast cancer, using a standardized protocol, and stored at −80° C. until use.

ELISA assays can use anti-HERV-K surface (SU) protein generated from expression vectors by SunnyBay Biotech (Fremont, Calif., USA). The bound antibodies are detected with a secondary antibody that binds to the serum antibodies and which are detected with a color reagent that reacts with the bound secondary antibody. The accessories for this device include reagents for antibody detection and color formation reagents for antibody visualization.

In a thirty-first embodiment, the ELISA kit can be a 96-well microwell plate coated with HERV-K antigen, which binds the antibodies in the patient's blood serum.

Reagents for detection of circulating serum antibodies against HERV-K are assembled for use in the clinical laboratory. Reagents for the assay are ready-to-use and pre-dispensed in sealed micro-ELISA 96-well plates. Sera to be assayed can be derived from women with female breast cancer, using a standardized protocol, and stored at −80° C. until use. ELISA assays use anti-HERV-K surface (SU) protein generated from expression vectors in the SunnyBay Biotech laboratory. The assay is summarized in FIG. 11.

### Exemplary Kit Contents for Serum Antibody Testing:

ELISA plate coated with antigen—96 well plate

Positive Control, 350 μl/vial

Negative Control, 350 μl/vial

Sample diluent, phosphate-buffered saline, 10.0 ml×1 bottle

Blocking buffer, 10.0 ml×1 bottle

HRP-conjugated anti-human IgG antibody, 1 ml/vial

HRP-conjugated anti-mouse IgG antibody, 1 ml/vial

TMB Peroxidase Substrate, 10 ml×1 bottle

TMB Peroxidase Substrate Solution B, 10 ml×1 bottle

Stop Solution, 1.0 M HCl, 10.0 ml×1 bottle

Manual [symbol] 1× paper. The kit label might list components suggested but not provided in the HERV-K ANTIBODY ELISA.

The conjugate pad of the assay device comprises a detection reagent or detectable marker. The detectable marker in the conjugate pad can bind the HERV antigen or the anti-HERV antibody that the sample pad receives. The conjugate pad acts to ensure uniform transfer of the detectable marker and the protein onto the assay membrane. The detectable marker comprises particles, luminescent labels, calorimetric labels, fluorescent labels, chemical labels, enzymes, radioactive labels, metal colloids, and chemiluminescent labels. In a thirty-second embodiment, gold colloidal spheres are used. other metal sols and latex microparticles are used. In a thirty-third embodiment, photostable, color tunable nanoparticles such as carbon, selenium, or quantum dots are used as detectable markers. These detectable markers provide colorimetric indicators for reporting whether the target HERV antigen or anti-HERV antibody is present. The size of the detectable markers is related to the porosity of the membrane. The markers are preferably sufficiently small to be transported along the membrane by the capillary action of the fluid. The amount of detectable marker present varies depending on the size and composition of the detectable marker, the composition of the membrane, and the level of sensitivity of the assay. The detectable marker binds to the HERV antigen or anti-HERV antibody to form a protein-detectable marker complex. The detectable marker comprises gold colloidal spheres. the detectable marker comprises a secondary protein. the secondary protein is an anti-HERV antibody. the secondary protein is conjugated to gold.

The measuring of the amount of the anti-HERV antibody target uses ELISA or ELISA-based assays. In a thirty-fourth embodiment, the measuring of the amount of HERV target uses qPCR, PCR, qRT-PCR, or RT-PCR, ligase chain reaction (LCR), ligase-mediated rapid amplification of cDNA ends, nucleic acid sequence-based amplification (NASBA) analysis, DNA or RNA sequencing, loop-mediated amplification, transcription-mediated amplification (TMA), or strand displacement amplification (SDA). The detection method comprises analysis of the HERV target in a tissue sample, e.g., using fluorescence in situ hybridization (FISH) or IHC). The anti-HERV antibody target is HERV protein or a HERV protein-derived polypeptide. The HERV target is HERV DNA, or HERV RNA. the target includes HERV-H, HERV-E, ERV-3, HERV-W, or another HERV subtype.

Assay kits can also be supplied for use with an anti-HERV antibody, such as a conjugated/labeled anti-HERV antibody, for the detection of a cellular activity or for detecting HERV peptides in a sample that includes a tissue or bodily fluid sample or host. In such diagnostic kits, and in kits for therapeutic uses described in this specification, an anti-HERV antibody typically can be provided in a lyophilized form in a container, either alone or with additional antibodies specific for a target cell or peptide. Typically, a pharmaceutical acceptable carrier, e.g., an inert diluent) or components thereof, such as a Tris, phosphate, or carbonate buffer, stabilizers, preservatives, biocides, biocides, inert proteins, e.g., serum albumin, or the like, also are included (typically in a separate container for mixing) and additional reagents (also typically in separate containers). In certain kits, a secondary antibody capable of binding to the anti-HERV antibody, which typically is present in a separate container, is also included. The second antibody is typically conjugated to a label and formulated in manner similar to the anti-HERV antibody of the invention. Using the methods described in this specification, anti-HERV antibodies can define subsets of cancer/tumor cells and characterize such cells and related tissues/growths.

The assay can be used kit for detection, diagnosis, or prognosis of cancer comprising a container comprising an anti-HERV antibody, and one or more reagents for detecting binding of the anti-HERV antibody to a HERV peptide in a sample that includes bodily fluids. Such a kit can further comprise anti-HERV antibody conjugates. Reagents can include, for example, fluorescent tags, enzymatic tags, or other detectable tags. The reagents can also include secondary or tertiary antibodies or reagents for enzymatic reactions, wherein the enzymatic reactions produce a product that can be visualized. The invention provides a diagnostic kit comprising one or more anti-HERV antibodies, of the invention in labeled or unlabeled form in suitable containers, reagents for the incubations for an indirect assay, and substrates or derivatizing agents for detection in such an assay, depending on the label. Control reagents and instructions for use optionally can be included.

### Composition of Matter

Antibody. The anti-HERV antibody can be mAb 6H5 or mAb 6E11, as described in this specification.

The anti-HERV antibodies of the invention can be used for diagnostic purposes. In a thirty-fifth embodiment, the anti-HERV antibodies described in this specification can be conjugated to a detection agent or label, making them suitable for diagnostic purpose. Anti-HERV antibody conjugated to a detection agent enables a direct detection of binding of the anti-HERV antibody to HERV, examples of “detection agent” or “label” are given in the following and reference to “anti-HERV antibody” in the following may where relevant also include reference to “anti-HERV antibody conjugated to a detection agent or label”. The term “diagnostic uses” includes also measuring the level of HERV in e.g. plasma, urine or expression levels of HERV in biopsies in relation to selecting patients for treatment or measuring the efficacy of a treatment as described above. Thus, in a further aspect, the invention relates to a diagnostic composition comprising an anti-HERV antibody wherein the diagnostic composition can in a thirty-sixth embodiment be used combined with an anti-HERV antibody conjugated to a detection agent or label of the invention.

The anti-HERV antibodies of the invention can be used in vivo or in vitro for detecting, diagnosing, and following progression of diseases wherein cells expressing HERV play an active role in the pathogenesis, by detecting levels of HERV, or levels of cells which contain HERV on their membrane surface. This can be achieved, for example, by contacting a sample to be assayed, optionally along with a control sample, with the anti-HERV antibody under conditions that allow for formation of a complex between the anti-HERV antibody and HERV. Complex formation is then detected, e.g., using an ELISA). When using a control sample along with the assay sample, complex is detected in both samples and any statistically significant difference in the formation of complexes between the samples indicates HERV in the assay sample.

The anti-HERV antibodies of the invention can also be used in a method for detecting HERV antigen, or a cell expressing HERV, in a sample comprising: contacting the sample with an anti-HERV antibody of the invention, under conditions that allow for formation of a complex between the antibody and HERV; and analyzing whether a complex was formed. The method is performed in vitro.

The anti-HERV antibodies of invention can also be used in methods for the identification of, and diagnosis of invasive cells and tissues, and other cells targeted by anti-HERV antibodies of the invention, and for the monitoring of the progress of therapeutic treatments, status after treatment, risk of developing cancer, cancer progression, and the like.

The anti-HERV antibodies of invention can be used in a method of diagnosing the level of invasive cells in a tissue. Such a method comprises forming an immunocomplex between an anti-HERV antibody and potential HERV-containing tissues, and detecting formation of the immunocomplex, wherein the formation of the immunocomplex correlates with the presence of invasive cells in the tissue. The contacting can be performed in vivo, using labeled isolated antibodies and standard imaging techniques, or can be performed in vitro on tissue samples.

The anti-HERV antibodies of the invention can also detect HERV-containing peptides and peptide fragments in any suitable biological sample by any suitable technique. Examples of conventional immunoassays provided by the invention include, without limitation, an ELISA, an RIA, FACS assays, plasmon resonance assays, chromatographic assays, tissue immunohistochemistry, Western blot, or immunoprecipitation using an anti-HERV antibody. Anti-HERV antibodies of the invention can detect HERV and HERV-fragments from humans. Suitable labels for the anti-HERV antibody or secondary antibodies used in such techniques include, without limitation, various enzymes, prosthetic groups, fluorescent materials, luminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ¹²⁵I, ¹³¹I, ³⁵S, and ³H.

The anti-HERV antibodies can also be used for assaying in a biological sample by a competition immunoassay utilizing HERV peptide standards labeled with a detectable substance and an unlabeled anti-HERV antibody. In such an assay, the biological sample, the labeled HERV peptide standards and the anti-HERV antibodies are combined and the amount of labeled HERV standard bound to the unlabeled anti-HERV antibody is determined. The amount of HERV peptide in the biological sample is inversely proportional to the amount of labeled HERV standard bound to the anti-HERV antibody.

The anti-HERV antibodies are useful in the in vivo imaging of tumors. In vivo imaging of tumors associated with HERV can be performed by any suitable technique. For example, 99Tc-labeling or labeling with another gamma-ray emitting isotope can label anti-HERV antibodies in tumors or secondary labeled, e.g., FITC-labeled) anti-HERV antibody:HERV complexes from tumors and imaged with a gamma scintillation camera, e.g., an Elscint Apex 409ECT device), typically using low-energy, high resolution collimator or a low-energy all-purpose collimator. Stained tissues can then be assessed for radioactivity counting as an indicator of the amount of HERV-associated peptides in the tumor. The images obtained by such techniques can assess biodistribution of HERV in a patient, mammal, or tissue, for example in using HERV or HERV-fragments as a biomarker for the presence of invasive cancer cells. Variations on this technique can include the use of magnetic resonance imaging (MRI) to improve imaging over gamma camera techniques. Such images can also be used for targeted delivery of other anti-cancer agents, examples of which are described in this specification, e.g., apoptotic agents, toxins, or CHOP chemotherapy compositions. Such images can also or alternatively serve as the basis for surgical techniques to remove tumors. Such in vivo imaging techniques can allow for the identification and localization of a tumor where a patient is identified as having a tumor, due to the presence of other biomarkers, metastases, etc., but the tumor cannot be identified by traditional analytical techniques.

The in vivo imaging and other diagnostic methods provided by the invention are useful in the detection of micrometastases in a human patient, e.g., a patient not previously diagnosed with cancer or a patient in a period of recovery/remission from a cancer. Carcinoma cancer cells, which may make up to 90% of all cancer cells, for example, were demonstrated to stain well with anti-HERV antibody compositions. Detection with monoclonal anti-HERV antibodies described in this specification may indicate carcinomas that are aggressive/invasive and also or indicate the feasibility of using related monoclonal anti-HERV antibody against such micrometastases.

The anti-HERV antibodies of the invention can be used in an in vivo imaging method wherein an anti-HERV antibody of the invention is conjugated to a detection-promoting radio-opaque agent, the conjugated antibody is administered to a host, such as by injection into the bloodstream, and the presence and location of the labeled antibody in the host is assayed. Through this technique and any other diagnostic method provided in this specification, the anti-HERV antibodies of the invention can be used in a method for screening for disease-related cells in a human patient or a biological sample taken from a human patient.

For diagnostic imaging, radioisotopes can be bound to an anti-HERV antibody either directly, or indirectly by using an intermediary functional group, such as radioisotopes and radio-opaque agents. Useful intermediary functional groups include chelators, such as ethylenediaminetetraacetic acid and diethylenetriaminepentaacetic acid. See for instance, U.S. Pat. No. 5,057,313.

Diagnostic methods can be performed using anti-HERV antibodies conjugated to dyes (such as with the biotin-streptavidin complex), contrast agents, fluorescent compounds or molecules and enhancing agents (e.g. paramagnetic ions) for magnetic resonance imaging (MRI). See, e.g., U.S. Pat. No. 6,331,175, which describes MRI techniques and preparing antibodies conjugated to a MRI enhancing agent. Such diagnostic/detection agents can be selected from agents for magnetic resonance imaging, and fluorescent compounds. To load an anti-HERV antibody with radioactive metals or paramagnetic ions, a person having ordinary skill in the biotechnological art react it with a reagent having a long tail to which are attached a multiplicity of chelating groups for binding the ions. Such a tail can be a polymer such as a polylysine, polysaccharide, or other derivatized or derivatizable chain having pendant groups to which can be bound chelating groups such as, e.g., porphyrins, polyamines, crown ethers, bisthiosemicarbazones, polyoximes, and like groups known to be useful for this purpose. Chelates can be coupled to anti-HERV antibodies using standard chemistries.

The invention provides diagnostic anti-HERV antibody conjugates, wherein the anti-HERV antibody is conjugated to a contrast agent (such as for magnetic resonance imaging, computed tomography, or ultrasound contrast-enhancing agent) or a radionuclide that may be, for example, a gamma-, beta-, alpha-, Auger electron-, or positron-emitting isotope.

Primers. Primers corresponding SEQ ID NO: 1 and SEQ ID NO: 2 are described in the SEQUENCE LISTING and elsewhere in this specification.

Panel of biomarkers. A panel biomarkers for use in testing for the presence of HERV or for increased HERV levels can include one or more of anti-human endogenous retrovirus (HERV) protein antibody; anti-HERV peptide antibody; anti-HERV Env surface (SU) protein antibody; anti-HERV Env surface (SU) peptide antibody; anti-HERV Env transmembrane (TM) protein antibody; anti-HERV Env transmembrane (TM) peptide antibody; anti-HERV Gag protein antibody; anti-HERV Gag peptide antibody; anti-HERV Pol protein antibody; anti-HERV Pol peptide antibody; anti-HERV NP9 protein antibody; anti-HERV NP9 peptide antibody; anti-HERV Rec protein antibody; anti-HERV Rec peptide antibody; anti-HERV multiple antigen peptide (MAP) antibody; HERV protein; HERV peptide; HERV Env surface (SU) protein; HERV Env surface (SU) peptide; HERV Env transmembrane (TM) protein; HERV Env transmembrane (TM) peptide; HERV Gag protein; HERV Gag peptide; HERV Pol protein; HERV Pol peptide; HERV NP9 protein; HERV NP9 peptide; HERV Rec protein; HERV Rec peptide; HERV RNA; HERV Env surface (SU) RNA; HERV Env transmembrane (TM) RNA; HERV Gag RNA; HERV Pol RNA HERV NP9 RNA; HERV Rec RNA; HERV DNA; HERV Env surface (SU) DNA; HERV Env transmembrane (TM) DNA; HERV Gag DNA; HERV Pol DNA; HERV NP9 DNA; HERV Rec DNA; immune checkpoint molecule PD-1 protein; immune checkpoint molecule PD-1 RNA; immune checkpoint molecule PD-L1 protein; immune checkpoint molecule PD-L1 RNA; immune checkpoint molecule PD-L2 protein; immune checkpoint molecule PD-L2 RNA; immune checkpoint molecule CTLA-4 protein; immune checkpoint molecule CTLA-4 RNA; immune checkpoint molecule LAG3 protein; immune checkpoint molecule LAG3 RNA; immune checkpoint molecule TIM3 protein; immune checkpoint molecule TIM3 RNA; immune checkpoint molecule CD27 protein; immune checkpoint molecule CD27 RNA; immune checkpoint molecule B7 family protein; immune checkpoint molecule B7 family RNA; HERV reverse transcriptase (RT) activity; a control (consensus) HERV immunosuppressive domain region (TMc) protein sequence; a control (consensus) HERV immunosuppressive domain region (TMc) RNA sequence; a control (consensus) HERV immunosuppressive domain region (TMc) protein with sequence GIHWQKNSARLWNSQSSIDQKLANQINDLRQTVIWMGDRLMSLEHRFQLQCDC (SEQ ID NO: 1); a HERV RNA sequence that codes for a control (consensus) (TMc) immunosuppressive domain region with protein sequence GIHWQKNSARLWNSQSSIDQKLANQINDLRQTVIWMGDRLMSLEHRFQLQCDC (SEQ ID NO: 1); a variant HERV immunosuppressive domain region (TMv) region protein with sequence; IGKRILQDCGIHNLVLIKNWQIKLIILDKLIWIGDRLMSLERRFQLQCDC (SEQ ID NO: 2); and an RNA sequence that codes for a variant HERV immunosuppressive domain region (TMv) region with protein sequence

(SEQ ID NO: 2)
IGKRILQDCGIHNLVLIKNWQIKLIILD
KLIWIGDRLMSLERRFQLQCDC.

HERV target can be a nucleic acid. The HERV target nucleic acid includes DNA or RNA. In a thirty-seventh embodiment, the HERV target nucleic acid genomic subtypes include gag, env, pol, NP9, or Rec nucleic acids, multiple antigen peptides (MAPs) consisting of peptides derived from the genomic subtypes, or a combination of one or more of these nucleic acids and peptides detected in sequence or in a series. The pattern of HERV subtype nucleic acid targets detected in a sample from a subject correspond to the diagnosis of an identifiable cancer in the subject; for example, the diagnosis, type, aggressiveness, or metastatic potential of cancer; the response to cancer therapy, resistance to cancer therapy, or recurrence of the cancer. The pattern of HERV genotypes present in a blood or tissue sample from a subject correspond to one or more infectious HERV virions in a sample. the pattern of HERV genotypes present in a sample from a subject correspond to one or more subtypes of HERV that have recombined to form infectious virions in a sample.

### Method of Antibody Titering

Anti-HERV-K antibody titer ELISA assay. The serum antibody titers against HERV-K env proteins are determined by ELISA using purified recombinant HERV-K fusion proteins (HERV-K antigen) produced at SunnyBay Biotech (Fremont, Calif., USA). ELISA assays are performed as described to detect anti-HERV-K antibody in human sera. See Hughes & Coffin J M. Nature Genetics, 29(4), 487-9 (2001); Larsson Kato, & Cohen, Current Topics Microbiol. Immunol., 148, 115-32 (1989). A 96-well ELISA plate is coated with HERV-K env fusion proteins (10 μg/ml). The protein used for coating is HERV-K env surface (SU) protein. Surface-fusion proteins are expressed from the pGEX-6p1 plasmid (K10G17) in BL-21 (DE3) in M15 Escherichia coli at 18° C. overnight with 1 mmol/L isopropyl-l-thio-B-d-galactopyranoside. Bacterial pellets are harvested, disrupted by lysozyme treatment followed by sonication, clarified by 0.2 μm filtration, and affinity purified with glutathione-Sepharose FF using an AKTA fast protein liquid chromatography (FPLC). The wells are blocked with 2% bovine serum albumin (BSA) in PBS-T (PBS-T: 1.0 g BSA added to 50 ml phosphate-buffered saline in 0.1% Tween-20). Sera (1:50 dilutions with phosphate-buffered saline (PBS)) is then added to the coated wells, followed by detection with horseradish peroxidase (HRP)-conjugated anti-human IgG antibody. The positive controls are serum from an anti-HERV-K-positive donor and an anti-HERV-K antibody with known antibody titers. The negative controls are serum from an anti-HERV-K-negative donor and blocking buffer. because of coating with the antigens, serum antibodies against HERV-K surface protein are determined. The inventors can use the same protein expression protocol and the same HERV-K fusion protein construct for all kits produced at SunnyBay Biotech (Fremont, Calif., USA), to maintain a consistent product coated on the ELISA plate.

### Method of Making Antibodies

The invention's HERV proteins are useful as antigens for generating antibodies that specifically bind to the HERV protein or derivatives. The anti-HERV antibodies can generate a secondary antibody that specifically binds to the anti-HERV antibody. These secondary antibodies are useful in detecting anti-HERV antibodies, such as those in samples from subjects being screened for cancer.

### Method of Diagnosis

In situ detection can be accomplished by removing a histological specimen from a patient and combining labeled anti-HERV antibodies (anti-HERV antibody conjugated to a detection agent), of the invention to such a specimen. The anti-HERV antibody of the invention can be provided by applying or by overlaying the labeled anti-HERV antibody of the invention to a biological sample. Using such a procedure, a person having ordinary skill in the biotechnological art can determine not only the presence of HERV or HERV-fragments but also the distribution of such peptides in the examined tissue, e.g., in assessing the spread of cancer cells). Using the invention, people having ordinary skill in the biotechnological art know that any of a wide variety of histological methods, such as staining procedures, can be modified to achieve such in situ detection.

### Method of Treatment

In a thirty-eighth embodiment, the HERV-positive cancer that expresses HERV is a metastatic tumor. The cancer is resistant to treatment with trastuzumab, cetuximab, lapatinib, erlotinib, or any other small molecule or antibody that targets a family of receptors. the cancer is resistant to treatment with chemotherapeutic agents that include anthracyclines (doxorubicin (Adriamycin®) and epirubicin (Ellence®)), Taxanes (docetaxel (Taxotere®) and paclitaxel (Taxon), carboplatin, cyclophosphamide (Cytoxan®), fluorouracil (5-FU), albumin-bound paclitaxel (nab-paclitaxel or Abraxane®), capecitabine (Xeloda®), eribulin (Halaven®), gemcitabine (Gemzar®), ixabepilone (Ixempra), liposomal doxorubicin (Doxil®), mitoxantrone, platinum (carboplatin, cisplatin), vinorelbine (Navelbine®), and combined chemotherapy drugs that include Adriamycin® and Cytoxan®; Adriamycin® and Taxotere®; Cytoxan®, methotrexate, and fluorouracil; fluorouracil, Adriamycin®, and Cytoxan®; and Cytoxan®, Adriamycin®, and fluorouracil.

### Improvements

The invention provides methods and compositions that embody the discovery that these autoantibodies against HERV show improved sensitivity and specificity for cancer versus normal serum, as compared to prior art cancer markers.

### Definitions

For convenience, the meaning of some terms and phrases used in the specification, examples, and appended claims, are listed below. Unless stated otherwise or implicit from context, these terms and phrases have the meanings below. These definitions are to aid in describing particular embodiments and are not intended to limit the claimed invention. Unless otherwise defined, all technical and scientific terms have the same meaning as commonly understood by a person having ordinary skill in the art to which this invention belongs. A term's meaning provided in this specification shall prevail if any apparent discrepancy arises between the meaning of a definition provided in this specification and the term's use in the art.

“Anti-HERV-K antibody” is an antibody which binds specifically to the antigen HERV-K, described in this specification.

“Antibody conjugate” is an anti-HERV antibody or an anti-HERV-K antibody, which is coupled to another moiety as described in the present application.

“Antibody” encompasses any immunoglobulin, e.g., IgG, IgM, IgA, IgE, IgD, etc., obtained from any vertebrate source. Included within this definition are polyclonal antibody, monoclonal antibody, and chimeric antibody.

“Antibody” or “antibody molecule” describes a functional component of serum and is often referred to either as a collection of molecules (antibodies or immunoglobulin) or as one molecule (the antibody molecule or immunoglobulin molecule). An antibody can bind to or reacting with a specific antigenic determinant (the antigen or the antigenic epitope), which can lead to induction of immunological effector mechanisms. An individual antibody is usually regarded as monospecific, and a composition of antibodies can be monoclonal (i.e., consisting of identical antibody molecules) or polyclonal (i.e., consisting of two or more different antibodies reacting with the same or different epitopes on the same antigen or even on distinct, different antigens). Each antibody has a unique structure that enables it to bind specifically to its corresponding antigen, and all natural antibodies have the same overall basic structure of two identical light chains and two identical heavy chains. Antibodies are also known collectively as immunoglobulins. Antibodies include single chain antibodies and binding fragments of antibodies, such as Fab, F(ab′)2, Fv fragments or single chain Fv (scFv) fragments, and multimeric forms such as dimeric IgA molecules or pentavalent IgM. In the present description and claims, references to an “antibody” or “antibodies” are therefore intended to encompass binding fragments and single chain antibodies, unless it is indicated otherwise or apparent from the context that this is not the case. Each heavy chain of an antibody typically includes a heavy chain variable region (VH) and a heavy chain constant region. The heavy chain constant region typically includes three domains, called CH1, CH2 and CH3. Each antibody light chain typically includes a light chain variable region (VL) and a light chain constant region. The light chain constant region typically includes a single domain, called CL. The VH and VL regions can be further subdivided into regions of hypervariability (“hypervariable regions”, which can be hypervariable in sequence or in structurally defined loops). The “hypervariable” regions found in the variable domains of an antibody primarily responsible for determining the antibody's binding specificity. These are also referred to as complementarity determining regions (CDRs), which are interspersed with regions that are more conserved, termed framework regions (FRs). Each of the heavy and light chains of an antibody contains three CDR regions, called CDR1, CDR2 and CDR3, of which CDR3 shows the greatest variability. Each VH and VL typically includes three CDRs and four FRs, arranged from the amino terminus to the carboxy terminus in the following order: FR1, CDR1, FR2, CDR2, FR3, CDR3, FR4. The amino acid residues in the variable regions are often numbered using a standardized numbering method known as the Kabat numbering scheme (Kabat et al, (1991) Sequences of Proteins of Immunological Interest, 5th Ed Public Health Service, National Institutes of Health, Bethesda, Md., USA), although other numbering schemes such as Chothia and IMGT also exist.

“Antigen,” “immunogen,” “antigenic,” “immunogenic,” “antigenically active,” “immunologic,” and “immunologically active” when made in reference to a molecule, refer to any substance capable of inducing a specific humoral immune response (including eliciting a soluble antibody response) or cell-mediated immune response (including eliciting a CTL response).

“B7 family” refers to inhibitory ligands with undefined receptors. The B7 family encompasses B7-H3 and B7-H4, both upregulated on tumor cells and tumor infiltrating cells. The complete hB7-H3 and hB7-H4 sequence can be found under GenBank Accession Nos. Q5ZPR3 and AAZ17406, respectively.

“Biological samples” include those obtained from an animal (including humans), including bodily fluid samples such as serum, plasma, blood, urine, cerebrospinal fluid (CSF), sputum, saliva, cell extract, tissue extract, etc., and solid samples such as tissue (such as biopsy material), cells, and the like.

“Bodily fluid” includes, but is not limited to serum, plasma, blood, urine, cerebrospinal fluid (CSF), sputum, saliva, cell extract, tissue extract, etc.

“Cancer antigen” refers to (i) tumor-specific antigens, (ii) tumor-associated antigens, (iii) cells that express tumor-specific antigens, (iv) cells that express tumor-associated antigens, (v) embryonic antigens on tumors, (vi) autologous tumor cells, (vii) tumor-specific membrane antigens, (viii) tumor-associated membrane antigens, (ix) growth factor receptors, (x) growth factor ligands, and (xi) any other antigen or antigen-presenting cell or material associated with a cancer.

“Cancer at risk for metastases” has the oncological art-recognized meaning of a cancer that can differentiate into a metastatic cancer. Such risk can be based on family history, genetic factors, type of cancer, environmental factors, etc.

“Cancer cell” and “tumor cell” has the oncological art-recognized meaning of a cell undergoing early, intermediate or advanced stages of multi-step neoplastic progression as described by Pitot et al., Fundamentals of Oncology, pp. 15-28. The features of early, intermediate and advanced stages of neoplastic progression were described using microscopy. Cancer cells at each of the three stages of neoplastic progression generally have abnormal karyotypes, including translocations, inversion, deletions, isochromosomes, monosomies, and extra chromosomes.

“Cancer” or “neoplasia” refers to a plurality of cancer cells.

“Carcinoma” has the oncological art-recognized meaning of a malignant new growth made up of epithelial cells infiltrating the surrounding tissues and give rise to metastases.

“Checkpoint blockade” has the oncological art-recognized meaning of a total or partial reduction, inhibition, or interference with or modulation of one or more checkpoint proteins.

“Chimeric antibody” has the biotechnological art-recognized meaning of an antibody that contains one or more regions from one antibody and one or more regions from one or more other antibodies. See, e.g.: U.S. Pat. No. 4,816,567 (Cabilly et al.); U.S. Pat. No. 4,978,745 (Shoemaker et al.); U.S. Pat. No. 4,975,369 (Beavers et al.); and U.S. Pat. No. 4,816,397 (Boss et al.). For a chimeric antibody, the non-human parts can be subjected to further alteration to humanize the antibody. The invention provides humanization of certain chimeric antibodies having murine variable region sequences.

“Combination therapy” “has the oncological art-recognized meaning of administration of each agent or therapy in a sequential manner in a regimen provides beneficial effects of the combination, and co-administration of these agents or therapies in a substantially simultaneous manner, such as in a single capsule having a fixed ratio of these active agents or in multiple, separate capsules for each agent. Combination therapy also includes combinations where individual elements can be administered at different times or by different routes but which act in combination to provide a beneficial effect by co-action or pharmacokinetic and pharmacodynamics effect of each agent or tumor treatment approaches of the combination therapy.

“Cytotoxic T Lymphocyte Associated Antigen-4 (CTLA-4)” has the biotechnological art-recognized meaning of a T cell surface molecule and is a member of the immunoglobulin superfamily. This protein downregulates the immune system by binding to CD80 and CD86. “CTLA-4” includes human CTLA-4 (hCTLA-4), variants, isoforms, and species homologs of hCTLA-4, and analogs having at least one common epitope with hCTLA-4. The complete hCTLA-4 sequence can be found under GenBank Accession No. P16410.

“Diagnose”, “diagnosis” or “diagnosing” has the oncological art-recognized meaning of the recognition of a disease by its signs and symptoms, e.g., resistance to conventional therapies), or genetic analysis, pathological analysis, histological analysis, and the like.

“Dysplastic cell” has the oncological art-recognized meaning of a cell in the intermediate stages of neoplastic progression is called a “dysplastic cell”. A dysplastic cell resembles an immature epithelial cell, is generally spatially disorganized within the tissue and loses its specialized structures and functions. During the intermediate stages of neoplastic progressions an increasing percentage of the epithelium becomes composed of dysplastic cells.

“Epitope” and “antigenic determinant” has the biotechnological art-recognized meaning of a structure on an antigen, which interacts with the binding site of an antibody or T cell receptor because of molecular complementarity. An epitope can compete with the intact antigen, from which it is derived, for binding to an antibody.

“Human endogenous retrovirus-K”, “HERV-K”, “HERV”, “human endogenous retrovirus”, “endogenous retrovirus”, and “ERV” include any variants, isoforms and species homologs of endogenous retroviruses naturally expressed by cells or are expressed on cells transfected with endogenous retroviral genes. HERV-K is transcriptionally active in germ cell tumors, melanoma, breast cancer cell lines (T47D), breast cancer tissues, and ovarian cancer. The inventors specifically identified HERV proteins and sequences in cancer cell lines, patient tumors and blood samples. See Wang-Johanning et al., Clin. Cancer Res. 7(6), 1553-60 (2001); Wang-Johanning et al., Int. J. Cancer, 120(1), 81-90 (2007). The inventors observed the expression of HERVs, especially HERV-K sequences, in breast, lung, prostate, ovarian, colon, pancreatic, and other solid tumors. See Wang-Johanning et al. Immunotherapeutic potential of anti-human endogenous retrovirus-K envelope protein antibodies in targeting breast tumors. J. Natl. Cancer Inst., 104(3), 189-210 (2012) and other scientific references. They also found that the expression of HERV-K env transcripts in breast cancer was specifically associated with the progression of neoplasia2d, such that later stage tumors increase the expression of HERV-K.

“Human endogenous retroviruses” are well-known in the biotechnological art as genomic repeat sequences, with many copies in the genome, such that approximately 8% of the human genome is of retroviral origin. Lander et al., Nature, 409(6822), 860-921 (2001). HERVs originated from thousands of ancient integration events which incorporated retrovirus DNA into germline cells. Typically, retroviruses lose infectivity because of the accumulation of mutations. Hence, these genes are predominantly silent and not expressed in normal adult human tissues, except during pathologic conditions such as cancer. The most biologically active HERVs are members of the HERV-K family, which is the only family to have the full complement of open reading frames typical of replication competent mammalian retroviruses. HERV-K expression is absent in normal human tissues, but as discussed throughout this specification HERV-K protein and anti-HERV-K antibodies are expressed in cancer patients and tumor tissues.

“Human monoclonal antibody” has the biotechnological art-recognized meaning of antibodies displaying a single binding specificity which have variable and constant regions derived from human germline immunoglobulin sequences. The human monoclonal antibodies can be generated by a hybridoma which includes a B cell obtained from a transgenic or transchromosomal non-human animal, such as a transgenic mouse, having a genome comprising a human heavy chain transgene and a light chain transgene, fused to an immortalized cell.

“Hyperplastic cell” has the oncological art-recognized meaning of a cell in the early stages of malignant progression and is characterized by dividing without control or at a rate greater than a normal cell of the same cell type in the same tissue.

“Immune cell” is a cell of hematopoietic origin and that plays a role in the immune response. Immune cells include lymphocytes, e.g., B cells and T cells), natural killer cells, and myeloid cells, e.g., monocytes, macrophages, eosinophils, mast cells, basophils, and granulocytes).

“Immune checkpoint” has the oncological art-recognized meaning of co-stimulatory and inhibitory signals that regulate the amplitude and quality of T cell receptor recognition of an antigen. The immune checkpoint can be an inhibitory signal. an “immune checkpoint protein” or “ICP” is an immune checkpoint that is a protein. The inhibitory signal can be the interaction between PD-1 and PD-L1. The inhibitory signal can be the interaction between CTLA-4 and CD80 or CD86 to displace CD28 binding. In certain embodiments the inhibitory signal is the interaction between LAG3 and MHC class II molecules. The inhibitory signal can be the interaction between TIM3 and galectin 9.

“Immunosuppressive domain” is the HERV-K immunosuppressive domain. Retroviral proteins contain a unique and conserved 17-amino acid sequence, termed CKS-17, which exhibits immunosuppressive and Th1-inhibiting properties. See, Haraguchi, Good, & Day-Good, Immunol. Res., 41(1), 46-55 (2008). CKS-17 suppresses cell-mediated immunity and inhibits natural killer cells, cytotoxic T lymphocytes, macrophage-mediated tumor lysis and production of IL-12, IL-2, gamma interferon (IFN-γ), and TNFα. This immunosuppressive domain (ISD) is also found in the HERV-H family where it stimulates CCL19 expression in tumor cells, which specifically recruits and expands immunoregulatory CD45-CD271+ mesenchymal stem cells (MSCs) in the TME. See Kudo-Saito et al., Cancer Res., 74(5), 1361-70 (2014). HERV-K has a unique immunosuppressive domain in its transmembrane region. Morozov, Dao Thi, & Denner, PLoS One, 8(8), e70399 (2013). This immunosuppressive domain sequence is contained in over 80% of the published HERV-K sequences. The HERV-K immunosuppressive domain has some homology to CKS-17. This immunosuppressive domain sequence is contained in over 80% of the published HERV-K sequences. Both the purified glycosylated HERV-K transmembrane env protein and carrier protein-conjugates of the immunosuppressive domain peptide dose-dependently inhibited peripheral blood mononuclear cell (PBMC) and murine splenocyte activation. Morozov, Dao Thi, & Denner, PLoS One, 8(8), e70399 (2013). Treatment of PBMC with the ISD-containing transmembrane protein of HERV-K increased expression of ten out of sixty-two cytokines on a microarray, including upregulation of immunosuppressive IL-10 and downregulation of immunoactivator IL-12 production. Morozov, Dao Thi, & Denner, PLoS One, 8(8), e70399 (2013). HERV-K proteins containing the immunosuppressive domain were also found to condition monocyte-derived dendritic cells, which became impaired in both recruiting T-cells into stable conjugates and in expansion of allogenic T-cells. Hummel et al., Eur. J. Immunol., 45(6), 1748-59 (2015). These results and data provide strong evidence that HERV-K transmembrane expression at the RNA and protein level is upregulated in breast cancer. This immunosuppressive transcript and protein can serve as a breast cancer immunosuppression biomarker.

“In vivo” means in a living organism.

“Isolated” when used in an isolated nucleotide or an isolated polypeptide, is a nucleotide or polypeptide that, by human intervention, exists apart from its native environment and is therefore not a product of nature. An isolated nucleotide or polypeptide can exist in a purified form or can exist in a non-native environment such as in a transgenic host cell.

“Native” or “wild type” means a gene naturally present in the genome of an untransformed cell. Similarly, when used in fora polypeptide, “native” or “wild type” refers to a polypeptide that is encoded by a native gene of an untransformed cell's genome.

“Isotype” refers to the immunoglobulin class (for instance IgG1, IgG2, IgG3, IgG4, IgD, IgA, IgE, or IgM) that is encoded by heavy chain constant region genes. The term “epitope” means a protein determinant capable of specific binding to an antibody. Epitopes usually consist of surface groupings of molecules such as amino acids or sugar side chains and usually have specific three dimensional structural characteristics, and specific charge characteristics. Conformational and nonconformational epitopes are distinguished because the binding to the former but not the latter is lost in the presence of denaturing solvents. The epitope can comprise amino acid residues directly involved in the binding (also called immunodominant component of the epitope) and other amino acid residues, which are not directly involved in the binding, such as amino acid residues effectively blocked by the specifically antigen binding peptide. The amino acid residue is within the footprint of the specifically antigen binding peptide.

“Lymphocyte Activation Gene-3 (LAG3)” has the oncological art meaning of an inhibitory receptor associated with inhibition of lymphocyte activity by binding to MHC class II molecules. This receptor enhances the function of Treg cells and inhibits CD8+ effector T cell function. The term “LAG3” includes human LAG3 (hLAG3), variants, isoforms, and species homologs of hLAG3, and analogs having at least one common epitope. The complete hLAG3 sequence can be found under GenBank Accession No. P18627.

“Metastasis” has the oncological art-recognized meaning of the spread of cancer cells from one organ or part thereof to surroundings thereof or another organs but is not limited thereto. Malignant cancer cells mainly have the ability for metastasis. Cancer cells exit from a primary cancer to the lymph system or blood system, circulate the blood vessels and grow in the normal tissues at other parts of the body. Cancer metastasis, as a typical feature of malignant cancer, is accounted for 90% of deaths due to cancer. Cancer metastasis inhibition is the inhibiting of the spread of cancer cells to other organs or the surroundings.

“Prevention” has the medical art-recognized of any activities to suppress or delay onset of cancer or cancer metastasis by the administration of the pharmaceutical composition of the invention and “treatment” means any action to improve symptoms caused by the cancer or cancer metastatic or to change symptoms by the cancer or cancer metastatic to more beneficial states.

“Metastatic” cancer cell refers to a cancer cell translocated from a primary cancer site (i.e., a location where the cancer cell initially formed from a normal, hyperplastic or dysplastic cell) to a site other than the primary site, where the translocated cancer cell lodges and proliferates.

“Modified” bases include tritylated bases and unusual bases such as inosine. A variety of modifications can be made to DNA and RNA; thus, “polynucleotide” embraces chemically, enzymatically, or metabolically modified forms.

“Monoclonal antibody” or “monoclonal antibody composition” has the biotechnological art-recognized meaning of preparing antibody molecules of single molecular composition. The monoclonal antibody or composition thereof can be drug conjugated antibodies according to the invention. A monoclonal antibody composition displays a single binding specificity and affinity for a particular epitope.

“Neoplastic” cell has the oncological art-recognized meaning of a cell in the advanced stages of neoplastic progression. Neoplastic cells typically invade adjacent tissues or are shed from the primary site and circulate through the blood and lymph to other locations in the body where they initiate secondary cancers.

“Nucleic acid” has the biotechnological art-recognized meaning of deoxyribonucleotides or ribonucleotides and polymers thereof in either single- or double-stranded form. The term encompasses nucleic acids containing known analogues of natural nucleotides with similar binding properties as the reference nucleic acid and are metabolized in a manner similar to naturally occurring nucleotides. Unless otherwise indicated, a particular nucleic acid sequence also implicitly encompasses conservatively modified variants thereof, e.g., degenerate codon substitutions, and complementary sequences and the sequences explicitly stated in the SEQUENCE LISTING. Specifically, a person having ordinary skill in the biotechnological art can achieve degenerate codon substitutions by generating sequences in which the third position of one or more selected or all codons is substituted with mixed-base or deoxyinosine residues, according to biotechnological art-recognized methods. (For arginine and leucine, modifications at the second base can also be conservative. The term nucleic acid is used interchangeably with gene, cDNA, and mRNA encoded by a gene. Polynucleotides can be composed of any polyribonucleotide or polydeoxribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons.

“Pharmaceutically acceptable” has the pharmacological art-recognized meaning of compounds, materials, compositions, or dosage forms which are, within the scope of sound medical judgment, suitable for contact with the tissues, organs, or bodily fluids of humans and animals without excessive toxicity, irritation, allergic response, or other problems or complications commensurate with a reasonable benefit/risk ratio.

“Polyclonal antibody” has the biotechnological art-recognized meaning of an immunoglobulin produced from more than a single clone of plasma cells.

“Monoclonal antibody” has the biotechnological art-recognized meaning of an immunoglobulin produced from a single clone of plasma cells.

“Polypeptide fragment” or “fragment”, when used referring to a reference polypeptide, refer to a polypeptide in which amino acid residues are deleted as compared to the reference, e.g., native) polypeptide itself, but where the remaining amino acid sequence is usually identical to the corresponding positions in the reference polypeptide. As mentioned above, sometimes such deletions can occur at the amino-terminus, carboxy-terminus of the reference polypeptide, or both.

“Polypeptide”, “protein”, and “peptide”, which are interchangeable in this specification, have biotechnological art-recognized meaning of a polymer of the protein amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” refers to peptides, polypeptides, and proteins, unless otherwise noted. Thus, exemplary polypeptides include gene products, naturally occurring proteins, homologs, orthologs, paralogs, fragments and other equivalents, variants, and analogs of the foregoing. The term “fusion polypeptide” and the like refer to a polypeptide comprised of two or more distinct polypeptides that are covalently bound. The isolated polypeptides comprise a fluorescent polypeptide, a compound-binding polypeptide, and a polypeptide target of the compound-binding (or voltage-sensing) polypeptide (polypeptide target), and variants or fragments of the polypeptides. The individual polypeptides that comprise the isolated polypeptide can be arranged in any fashion. Some isolated polypeptides can be, from the N-terminus to C-terminus, the compound-binding polypeptide, the fluorescent polypeptide, and the polypeptide target. The isolated polypeptide can be, from the C-terminus to N-terminus, the compound-binding polypeptide, the fluorescent polypeptide, and the polypeptide target. The disclosed polypeptides include fusion polypeptides.

“Pre-neoplastic” cells have oncological art-recognized meaning of “hyperplastic” and “dysplastic” cells.

“Programmed Death Ligand-1 (PD-L1)” has the oncological art-recognized meaning of one of two cell surface glycoprotein ligands for PD-1 (the other being PD-L2) that downregulates T cell activation and cytokine secretion upon binding to PD-1. The term “PD-L1” includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

“Programmed Death-1 (PD-1)” receptor refers to an immuno-inhibitory receptor belonging to the CD28 family. PD-1 is expressed predominantly on previously activated T cells in vivo, and binds to two ligands, PD-L1 and PD-L2. The term “PD-1” includes human PD-1 (hPD-1), variants, isoforms, and species homologs of hPD-1, and analogs having at least one common epitope with hPD-1. The complete hPD-1 sequence can be found under GenBank Accession No. AAC51773.

“Purified,” “isolated,” and grammatical equivalents thereof refer to the reduction in the amount of at least one undesirable component (such as cell type, protein, or nucleic acid sequence) from a sample, including a reduction by any numerical percentage of from 5% to 100%, such as from 10% to 100%, from 20% to 100%, from 30% to 100%, from 40% to 100%, from 50% to 100%, from 60% to 100%, from 70% to 100%, from 80% to 100%, and from 90% to 100%. Thus purification results in “enrichment,” i.e., an increase in the amount of a desirable cell type, protein or nucleic acid sequence in the sample. The invention contemplates purified or isolated sialylated glycans.

“Recombinant antibody” has the biotechnological art-recognized meaning of an antibody expressed from a cell or cell line transfected with an expression vector (or possibly more than one expression vector, e.g. two expression vectors) comprising the coding sequence of the antibody, where the coding sequence rarely is associated with the cell.

“Reverse transcriptase” is an enzyme, encoded by the pol gene from retroviruses, that allows the virus to convert its RNA-based genome into DNA via reverse transcription. As a retrovirus, HERV-K relies on reverse transcriptase (RT) for successful integration into the host genome after infection. If these infection and stable integration events occur in germ cells, the viral sequences are transmitted horizontally from parent to child. This is generally understood to be the mechanism by which HERV retroviral elements exist as they do today in the human genome. HERV-K is of great interest among all the retroviral elements found in the human genome because of its relatively recent integration, transcriptional activity, and existence of open reading frames at various loci across the human genome for many of its proteins. Functional Env and reverse transcriptase proteins of a retrovirus are prerequisites for virulence. These data provide evidence that the enzyme activity of reverse transcriptase, as a stand-alone enzyme or as part of an HERV-K viral particle, is a blood biomarker of human cancer.

“Sample” and “specimen” are used in their broadest sense to include any composition obtained or derived from biological or environmental source, and sampling devices, e.g., swabs) that are brought into contact with biological or environmental samples.

“Sensitivity” of a method or molecule for disease, such as “sensitivity for cancer” which is interchangeably used with “cancer sensitivity,” refers to the proportion, e.g., percentage, fraction, etc., of positives (i.e., individuals having cancer) that are correctly identified as such, e.g. the percentage of people with cancer who are identified as having the condition. Sensitivity can be calculated according to the following equation: Sensitivity=number of true positives/(number of true positives+number of false negatives). Sensitivity is expressed (together with specificity) as a statistical measure of the performance of a binary classification test, such as using AUC of a ROC curve, as discussed above regarding specificity. The cancer is deemed to express a low level of HERV when the cancer was determined not to overexpress HERV based on the following characterization of the cancer: (a) a first determination of a level of HERV in a assay specimen comprising cells of the cancer is reported as negative, or (b) a first determination of a level of HERV in a assay specimen comprising cells of the cancer is reported as equivocal, and a second determination of a level of HERV in a assay specimen comprising cells of the cancer is reported as equivocal or negative. the determination of the level of HERV in the assay specimen is reported as negative when the level of HERV in the assay specimen is characterized as (i) (1) immunohistochemistry IHC 1+, wherein the level of HERV in the assay specimen is characterized as IHC 1+ when the assay specimen exhibits an incomplete HERV membrane staining that is faint/barely perceptible and within greater than 10% of the invasive tumor cells, wherein the staining is readily appreciated using a low-power objective; (2) IHC 0, wherein the level of HERV in the assay specimen is characterized as IHC 0 when the assay specimen exhibits no HERV staining observed, wherein the lack of staining is readily appreciated using a low-power objective, or a HERV membrane staining that is incomplete and is faint/barely perceptible and within less than or equal to 10% of the invasive tumor cells, wherein the staining is readily appreciated using a low-power objective; or (ii) in situ hybridization (ISH) negative, wherein the level of HERV in the assay specimen is characterized as ISH negative when the assay specimen exhibits a single-probe average HERV copy number of less than 4.0 signals per cell. the determination of the level of HERV in the assay specimen is reported as equivocal when the level of HERV in the assay specimen is: (i) IHC 2+, wherein the level of HERV in the assay specimen is characterized as IHC 2+ when the assay specimen exhibits (1) a circumferential HERV membrane staining that is incomplete or weak/moderate and within greater than 10% of invasive tumor cells, wherein the staining is observed in a homogenous and contiguous population, and wherein the staining is readily appreciated using a low-power objective; or (2) a complete and circumferential HERV membrane staining that is intense and within less than or equal to 10% of invasive tumor cells, wherein the staining is readily appreciated using a low-power objective; or (ii) ISH equivocal, wherein the level of HERV in the assay specimen is ISH equivocal when the assay specimen exhibits (1) a single-probe ISH average HERV copy number of greater than or equal to 4.0 and less than 6.0 signals/cell, wherein the copy number is determined by counting at least 20 cells within the area and is observed in a homogenous and contiguous population.

“Specific binding,” “binding specificity,” and equivalents thereof when made referring to the binding of a first molecule, such as a polypeptide, glycoprotein, nucleic acid sequence, polysaccharide, antigen, etc., to a second molecule, such as a polypeptide, glycoprotein, nucleic acid sequence, polysaccharide, antibody etc., refer to the preferential interaction between the first molecule with the second molecule as compared to the interaction between the second molecule with a third molecule. Specific binding is a relative term that does not require absolute specificity of binding. The term “specific binding” does not require that the second molecule interact with the first molecule absent an interaction between the second molecule and the third molecule. Rather, it is sufficient that the level of interaction between the first molecule and the second molecule is higher than the level of interaction between the second molecule with the third molecule. “Specific binding” of a first molecule with a second molecule also means that the interaction between the first molecule and the second molecule depends upon the presence of a particular structure on or within the first molecule. When a second molecule is specific for structure “A” that is on or within a first molecule, the presence of a third nucleic acid sequence containing structure A reduces the amount of the second molecule which is bound to the first molecule.

“Specificity” of a method or molecule for disease, such as “specificity for cancer” which is interchangeably used with “cancer specificity”, refers to the proportion, e.g., percentage, fraction, etc., of negatives, i.e., healthy individuals not having disease, that are correctly identified, i.e., the percentage of healthy subjects who are correctly identified as not having disease. Specificity can be calculated according to the following equation: Specificity=number of true negatives/(number of true negatives+number of false positives. Specificity is expressed together with sensitivity as a statistical measure of the performance of a binary classification test, such as using a Receiver Operator Characteristic (ROC) curve. For any assay, there is a trade-off between specificity and sensitivity. For cancer screening assays of human subjects, it is undesirable to risk falsely identifying healthy people as having cancer (low specificity), due to the high costs. These costs are both physical (unnecessary risky procedures) and financial. This trade-off can be represented graphically using a ROC curve.

“Receiver Operator Characteristic curve” and “ROC curve” mean to a plot of the true positive rate (AKA sensitivity) versus true negative rate (AKA 1-specificity). The measured result of the assay is represented on the x axis while the y axis represents the number of control, e.g., healthy, or case, e.g., cancer, subjects. For any cut point (each point along the x axis) a sensitivity and specificity of the assay can be measured. The range of sensitivity and specificity for any assay can range from 0% to 100%, depending on the selected cut point.

“Area under the curve” (“AUC”) for a Receiver Operator Characteristic (ROC) curve plot is equal to the probability that a classifier ranks a randomly chosen positive instance higher than a randomly chosen negative one. Thus, AUC is a general measure of an assay ability to discriminate between case, e.g., cancer, and control, e.g., healthy, subjects. Random chance would generate an AUC of 0.5. Useful assays can have AUC's greater than 0.50, including any value from 0.51 to 1.00. The AUC is used as the standard measure of an assays specificity or sensitivity.

“Subject in need of” reducing one or more symptoms of a disease, e.g., in need of reducing cancer metastasis or in need of reducing one or more symptoms of cancer, includes a subject that exhibits or is at risk of exhibiting one or more symptoms of the disease. Subjects may be at risk based on family history, genetic factors, environmental factors, etc. This term includes animal models of the disease. Thus, administering a composition that reduces a disease or which reduces one or more symptoms of a disease to a subject in need of reducing the disease or of reducing one or more symptoms of the disease includes prophylactic administration of the composition (i.e., before the disease or one or more symptoms of the disease are detectable) or therapeutic administration of the composition, i.e., after the disease or one or more symptoms of the disease are detectable.

“Subject” and “animal” that may benefit from the invention's methods interchangeably includes any multicellular animal, preferably a mammal.

“Synergy” or “synergistic effect” with regard to an effect produced by two or more individual components refers to a phenomenon in which the total effect produced by these components, when utilized in combination, is greater than the sum of the individual effects of each component acting alone.

“T cell cytotoxicity” includes any immune response that is mediated by CD8+ T cell activation. Exemplary immune responses include cytokine production, CD8+ T cell proliferation, granzyme or perforin production, and clearance of an infectious agent.

“T Cell Membrane Protein-3 (TIM3)” is an inhibitory receptor involved in the inhibition of lymphocyte activity by inhibition of TH1 cells responses. Its ligand is galectin 9, which is upregulated in various types of cancers. “TIM3” includes human TIM3 (hTIM3), variants, isoforms, and species homologs of hTIM3, and analogs having at least one common epitope. The complete hTIM3 sequence can be found under GenBank Accession No. Q8TDQo.

“T cell” refers to a CD4+ T cell or a CD8+ T cell. The term T cell encompasses TH1 cells, TH2 cells and TH17 cells.

“Treat”, “treating”, “treatment” and grammatical equivalents refers to combating a disease or disorder, as for example in the management and care of a patient. Treating a disease, e.g., cancer, metastasis, etc., can includes reducing one or more symptoms of the disease.

“Tumor-associated antigen immunological test system” has the U.S. Food & Drug Administration meaning of a device that consists of reagents used to qualitatively or quantitatively measure, by immunochemical techniques, tumor-associated antigens in serum, plasma, urine, or other body fluids. This device is intended as an aid in monitoring patients for disease progress or response to therapy or for the detection of recurrent or residual disease. See 21 C.F.R. 866.6010.

“Variant” refers to an amino acid sequence that is different from the reference polypeptide sequence by the location or type of one or more amino acids. Thus, a variant can include one or more amino acid substitutions.

Subject “at risk” for disease (such as cancer) refers to a subject that is predisposed to contracting or expressing one or more symptoms of the disease. This predisposition can be genetic, e.g., a particular genetic tendency to expressing one or more symptoms of the disease, such as heritable disorders, etc., or due to other factors, e.g., environmental conditions, exposures to detrimental compounds, including carcinogens, present in the environment, etc. The term subject “at risk” includes subjects “suffering from disease,” i.e., a subject that is experiencing one or more symptoms of the disease. The invention should not be limited to any particular signs or symptoms, but rather should encompass subjects that are experiencing any range of disease, from sub-clinical symptoms to full-blown disease, wherein the subject exhibits at least one of the indicia, e.g., signs and symptoms, associated with the disease.

Guidance from Materials and Methods

A person of ordinary skill in the biotechnological art or the oncological art can use these materials and methods as guidance to predictable results when making and using the invention:

Alignment of sequences for comparison. An optimal alignment of sequences for comparison can be conducted, e.g., by the local homology algorithm of Smith & Waterman, Adv. Appl. Math., 2, 482 (1981), by the homology alignment algorithm of Needleman & Wunsch, J. Mol. Biol. 48, 443 (1970), by the search for similarity method of Pearson & Lipman, Proc. Natl. Acad. Sci. U.S.A. 85, 2444 (1988), by computerized implementations of these algorithms (GAP, BESHERV-KIT, FASTA, and HERV-KASTA in the Wisconsin Genetics Software Package, Genetics Computer Group, 575 Science Dr., Madison, Wis., USA), or by visual inspection.

Determining percent sequence identity and sequence similarity. An algorithm that is suitable for determining percent sequence identity and sequence similarity is the BLAST algorithm, which is described in Altschul et al., J. Mol. Biol., 215, 403-410 (1990). Software for performing BLAST analyses is publicly available through the National Center for Biotechnology Information website.

Anti-HERV-K antibody titer ELISA assay. An exemplary protocol for the anti-HERV-K antibody titer ELISA assay is as follows:

### Coating of Plates:

1. Coat 96-well ELISA plates with 1 ug/ml of HERV-K antigen and incubate overnight at 4° C. The antigen is prepared as a 1.0 ug/ml stock solution, and 100 μl of this stock solution is added per well.

2. Prepare blocking buffer: 2% BSA in PBS-T (PBS-T: 1.0 g BSA added to 50 ml phosphate-buffered saline in 0.1% Tween-20).

3. Decant the antigen solution from the plate and wash 3 times with phosphate-buffered saline.

4. Add 200 ul of blocking buffer to each well. Incubate at room temperature for one hour on a platform rotator.

5. Decant blocking buffer from the plate.

6. Air-dry the plates overnight and package in a light-proof vapor-loc foil pouch that is heat-sealable and equipped with a zip lock. The pouch contains a 2-gram indicating desiccant pack. Leave the plates out another 24 hours prior to refrigeration to continue the desiccation process inside the sealed pouch before storage at 4° C.

### Protocol for End-User:

1. Dilute patient serum 1:50 in blocking buffer. Dilute positive and negative control serum 1:50 in blocking buffer and use phosphate-buffered saline as a negative reagent control.

2. Add 100 ul of diluted sera, positive control, or negative control to wells of a 96-well ELISA plate.

3. Incubate at room temperature for one hour on a platform rotator.

4. Decant diluted sera from plate and wash 5-6 times with blocking buffer.

5. Secondary antibody: Add 100 ul/well of HRP-conjugated anti-human IgG antibody (1:5000 dilution in blocking buffer) to all the patient serum samples and negative control. Add 100 ul/well of HRP-conjugated anti-mouse IgG antibody (1:5000 dilution in blocking buffer) to the positive control sample.

6. Incubate for one hour at room temperature on a platform rotator.

7. Decant the diluted HRP-conjugated secondary antibodies from the plate.

8. Wash five times with blocking buffer.

9. To read absorbance: Mix equal amounts of 3,3′,5,5′-tetramethylbenzidine (TMB) Peroxidase Substrate and TMB Peroxidase Substrate Solution B and add 100 μl/well. Incubate for thirty minutes at room temperature, add 100 μl/well of 1.0 M HCL as a Stop Solution, and read absorbance at 450 nm.

Use of a control sample. The invention's methods can use a control sample. The control sample is from a tissue lacking cancer or blood from a human subject lacking cancer. This is exemplified by a control sample from the same or different subject, such as a subject matched for gender or age, subject having a benign tumor, etc.

Assay methods. The level of HERV in the assay specimen is characterized as IHC 2+ when the assay specimen exhibits (1) a circumferential HERV membrane staining that is incomplete or weak/moderate and within greater than 10% of invasive tumor cells, wherein the staining is observed in a homogenous and contiguous population, and wherein the staining is readily appreciated using a low-power objective; or (2) a complete and circumferential HERV membrane staining that is intense and within less than or equal to 10% of invasive tumor cells, wherein the staining is readily appreciated using a low-power objective. a level of HERV in a assay specimen comprising cells of the cancer is characterized as IHC 1+. the level of HERV in the assay specimen is characterized as IHC 1+ when the assay specimen exhibits an incomplete HERV membrane staining that is faint/barely perceptible and within greater than 10% of the invasive tumor cells, wherein the staining is readily appreciated using a low-power objective. a level of HERV in a assay specimen comprising cells of the cancer is characterized as IHC 0. the level of HERV in the assay specimen is characterized as IHC 0 when the assay specimen exhibits no HERV staining observed, wherein the lack of staining is readily appreciated using a low-power objective, or a HERV membrane staining that is incomplete and is faint/barely perceptible and within less than or equal to 10% of the invasive tumor cells, wherein the staining is readily appreciated using a low-power objective.

The HERV-positive cancer that expresses HERV is a PD-L1-positive cancer. the HERV-positive cancer overexpresses PD-L1 relative to expression of PD-L1 in analogous noncancerous cells of the same tissue type as the cancer. the HERV-positive cancer is deemed to overexpress PD-L1 when a assay specimen comprising cells of the cancer expresses a detectable level of PD-L1 above background. the cancer is resistant to PD-L1 blockade with an anti-PD-L1 therapy. the anti-PD-L1 therapy is an anti-PD-L1 antibody. the anti-PD-L1 antibody is atezolizumab. the cancer is resistant to PD-1 blockade with an anti-PD-1 therapy. the anti-PD-1 therapy is an anti-PD-1 antibody. the anti-PD-1 antibody is pembrolizumab.

The HERV-positive cancer that expresses HERV includes breast cancer, triple negative breast cancer, ovarian cancer, prostate cancer, pancreatic cancer, colorectal cancer, cervical cancer, soft tissue sarcoma, or melanoma.

### Sequence Listing Free Text

TMc (with immunosuppressive domain (ISD)) primer:

(SEQ ID NO: 1)
GIHWQKNSARLWNSQSSIDQKLANQINDL
RQTVIWMGDRLMSLEHRFQLQCDC-TM.

TMv (without immunosuppressive domain (ISD)) primer:

(SEQ ID NO: 2)
IGKRILQDCGIHNLVLIKNWQIKLIILDK
LIWIGDRLMSLERRFQLQCDC-TM.

The variant sequence is found in at least melanoma patients.

K10 sense primer:
(SEQ ID NO: 3)
5′-AGAAAAGGGCCTCCACGGAGATG-3′).
primer
K10 antisense primer:
(SEQ ID NO: 5)
3′-ACTGCAATTAAAGTAAAAATGAA-5′.
K22 sense primer:
(SEQ ID NO: 4)
5′-GTATGCTGCTTGCAGCCTTGATGAT-3′.

### EXAMPLES

The following Examples are merely illustrative and are not intended to limit the scope or content of the invention in any way.

### Example 1

### Humoral Immune Responses Against HERV Transmembrane (TM) Envelope Domain in Breast Cancer Patients

The inventors determined that HERV-K transmembrane-specific immune response was induced in patients with breast cancer. Antibodies against the transmembrane domain were present in sera from patients with breast cancer.

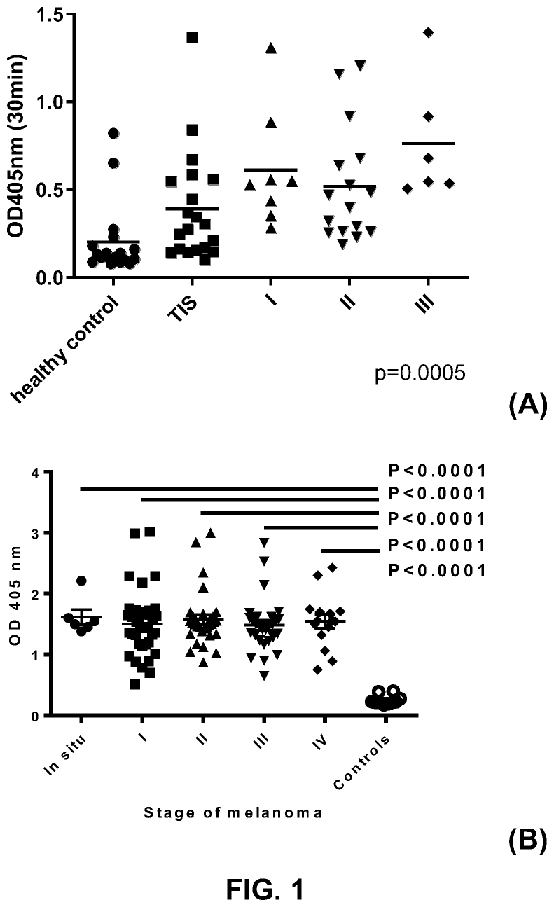

To evaluate the correlation between the specific humoral immune response and severity of the disease, the inventors performed ELISA with sera from breast cancer patients at different stages. The ELISA results showed that titer of transmembrane-specific IgG antibodies was elevated in sera from patients compared to healthy donors. 46% of breast cancer patients (23 in 50) were positive for antibodies against HERV-K transmembrane envelope protein (OD>0.5). All of the assayed patients with stage III breast cancer were positive for transmembrane-specific antibodies. See FIG. 1. Around 11% of healthy donors (2 in 18) were positive for transmembrane antigen. The mean absorbance was below 0.5.

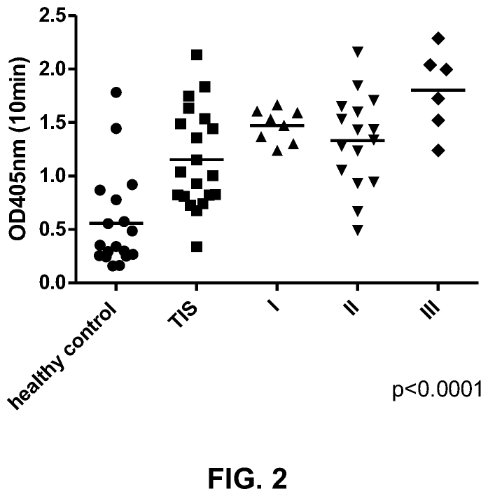

FIG. 1(A) shows an increased tendency for anti-transmembrane IgG antibody titers in patients with breast cancer at higher stage, even though no significant difference was observed except between patients in groups with cancer in situ and stage III breast cancer. Similar results were determined in the same cohort of breast cancer patients. See FIG. 2.

This EXAMPLE shows that the transmembrane protein of HERV-K is overexpressed in breast cancer patient primary breast cancer tumor cells (DCIS T and IDC T), but not in uninvolved normal breast cells (IDC N) using anti-TM antibody. The inventors assayed the expression of HERV-K transmembrane envelope protein in primary cells obtained from human breast cancer patient samples, ductal carcinoma in situ (DCIS) and invasive ductal carcinoma (IDC). Cells were fixed, permeabilized, and stained for HERV-K transmembrane envelope protein expression with mouse anti-transmembrane serum and with mock serum. HERV-K transmembrane protein is overexpressed in breast cancer samples relative to levels in non-involved adjacent breast tissue samples. Clinical samples from invasive ductal carcinoma and ductal carcinoma in situ breast cancer patient tumor cells overexpressed the HERV-K transmembrane protein. By contrast, the non-involved tissue adjacent to invasive ductal carcinoma tissue showed no transmembrane protein expression. Five human breast cancer cell lines showed much stronger expression of HERV-K transmembrane mRNA compared to that of the normal breast cell line MCF-10A.

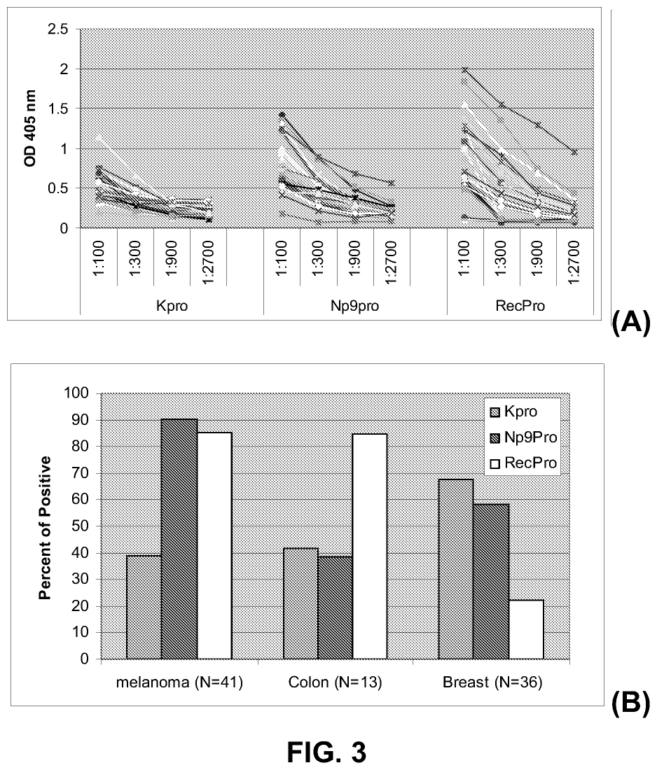

Similar results were determined for the IgG responses against HERV-K surface domain produced in melanoma patients. See FIG. 1(B). Expression of not only HERV-K but also the HERV-K splice variants Np9 and Rec was shown in melanoma patients. See FIG. 3.

The inventors detected a variant immunosuppressive domain in a melanoma patient (TMv), but the variant was not found in breast cancer patients, especially from viral particles obtained from an invasive ductal carcinoma breast cancer patient's plasma after infecting 293T cells. The inventors cloned transmembrane proteins containing the TMv immunosuppressive domain and the consensus immunosuppressive domain (TMc) into vectors and expressed the two immunosuppressive domains in 4T1 (4T1_K) and B16F10 cells. See the SEQUENCE LISTING.

### Example 2

### Humoral Immune Responses Against HERV Multiple Antigen Peptides (MAPs) in Breast Cancer Patients

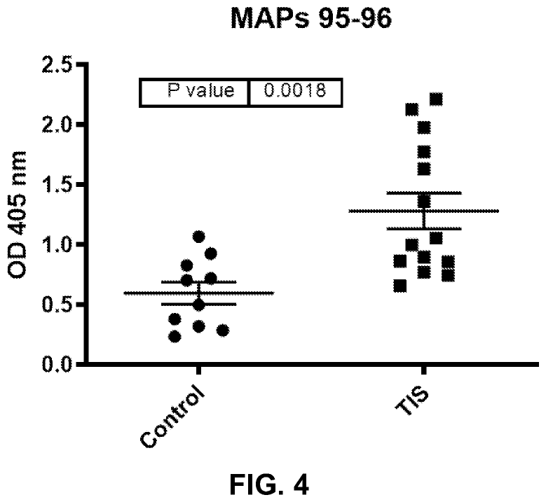

The HERV-K MAP-specific immune response was induced in patients with breast cancer. Antibodies against these multiple antigen peptides were present in sera from patients with breast cancer. To evaluate the correlation between the specific humoral immune response and severity of the disease, the inventors performed ELISA with sera from breast cancer patients at different stages. Overall, the titer of MAP-specific IgG antibodies was elevated in sera from patients with TIS [Neoplasm of breast primary tumor staging category Tis (DCIS): Ductal carcinoma in situ] compared with control subjects without cancer, using a HERV-K MAP peptide as antigen coated on the ELISA plate. See FIG. 4.

### Example 3

### Evaluating Baseline Immune Status in Relation to HERV Status in Breast Cancer Patients: Combined HERV and Immune Checkpoint Assays

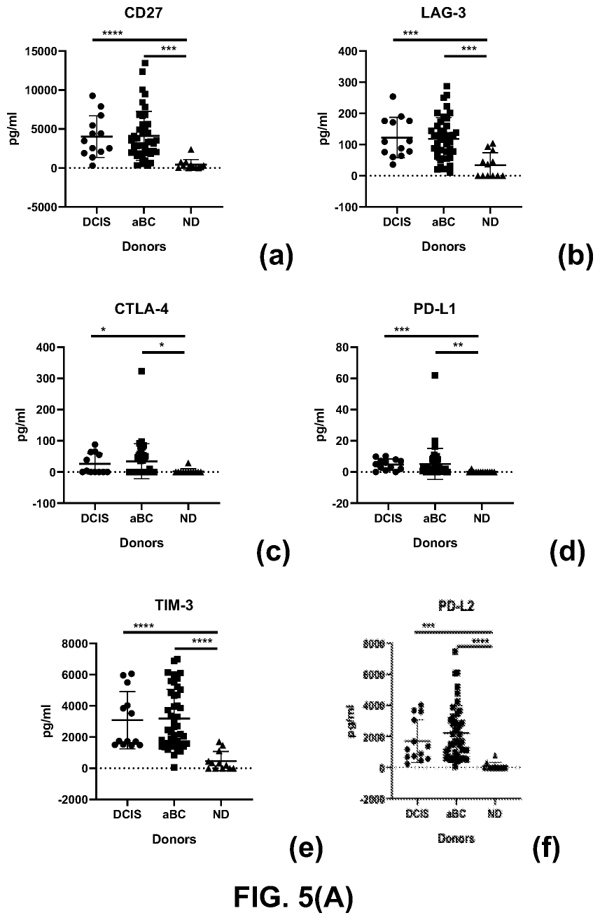

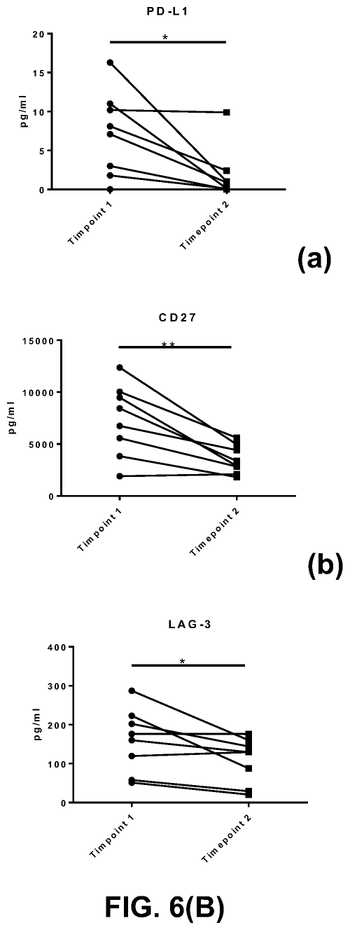

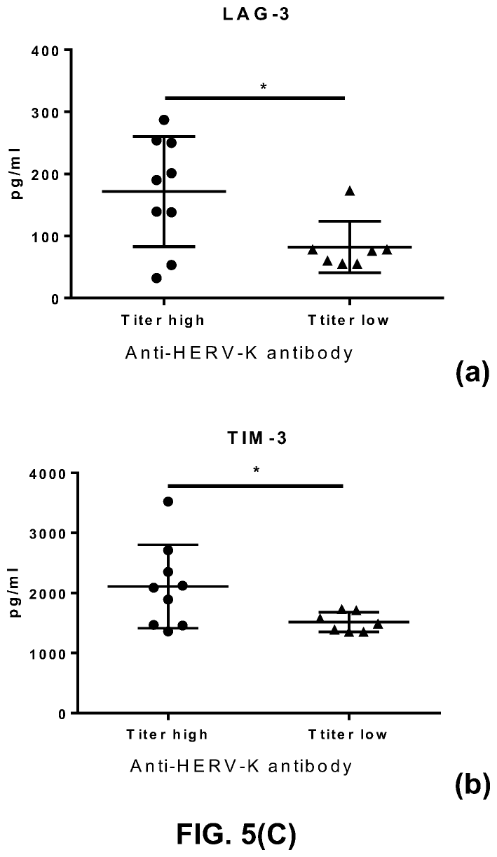

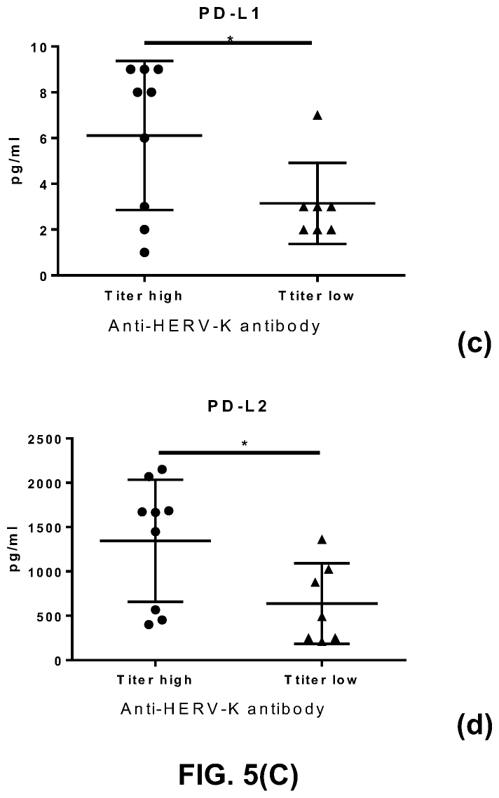

Expression of soluble immune checkpoint proteins was determined by Luminex assay in breast cancer patients, including ductal carcinoma in situ (DCIS) and aggressive breast cancer vs. normal donors. A striking and surprising finding was a significantly enhanced expression of six circulating immune checkpoint proteins in the plasma of breast cancer patients. See FIG. 5A. A further finding was a marked drop in immune checkpoint protein levels in patients at 6 (FIG. 5B; Timepoint 2) or eighteen months (data not shown) post-surgery vs. pre-surgery (Timepoint 1). Importantly, a positive association between soluble immune checkpoint protein molecule levels and HERV-K antibody titers induced by HERV-K expression in the tumor was observed. See FIG. 5(C). These results support the idea that HERV-K antibody titers would influence immune checkpoint protein levels in breast cancer. This correlation suggests that the expression of HERV-K can control immune responses for breast cancer patients.

### Example 4

### Immunohistochemistry Detection of HERV Envelope Protein Expression

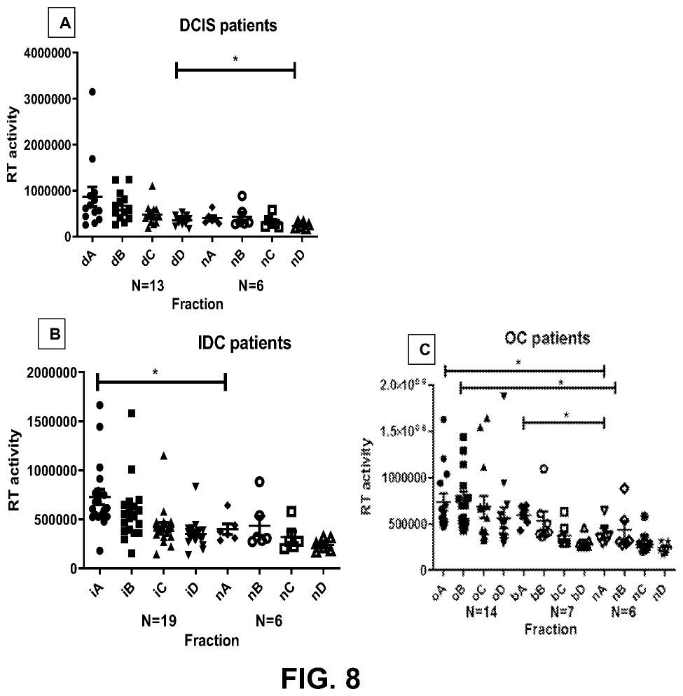

Immunohistochemistry was performed on 5-μm formalin-fixed paraffin-embedded tissue sections using standard protocols and the VECTASTAIN Elite ABC Kit. The expression of antigens was evaluated with the primary monoclonal antibody (mAb) 6H5 mAb for HERV-K envelope protein (Env) detection. See FIG. 8. The slides were baked in an oven at 60° C. for one hour, then deparaffinized with 100% xylene at room temperature for one minute, then hydrated in a graded alcohol series consisting of two 30-second dips each in 100% and 95% ethyl alcohol diluted in water (total volume is 5 mL) at room temperature, and then hydrated in water. Sections were incubated in 3% hydrogen peroxidase in water at room temperature for ten minutes to block endogenous peroxidase activity. After washing the slides for five minutes in water, blocking solution (four drops of stock horse serum in 10 mL of 1× phosphate-buffered saline; horse serum was provided in the VECTASTAIN Elite ABC Kit) is added, and the slides are incubated at room temperature for 30 minutes. Slides were then incubated with 6H5 mAb (1 μg in 150 μL normal serum buffer per slide) at 4° C. for sixteen hours. After three washes with 1× phosphate-buffered saline, slides were incubated for thirty minutes with anti-mIgG-HRP secondary antibody (1:600 dilution) in blocking buffer. After three washes with 1× phosphate-buffered saline, slides are incubated in 3,3-diaminobenzidine for five minutes and counterstained with hematoxylin. Envelope expression is categorized by intensity (0=absent; 1=weak; 2=moderate; 3=strong) and distribution (percent tumor positive for envelope). Intensity and distribution scores were multiplied to obtain the final score (0-300) for Env expression in a tumor.

The inventors detected of HERV-K env protein expression in one 59-year-old female diagnosed with invasive ductal carcinoma. The inventors detected of HERV-K env protein expression in regions of invasive ductal carcinoma, ductal carcinoma in situ, uninvolved epithelial cells, and normal cells. Positive staining was detected in tumor epithelial cells from invasive ductal carcinoma and ductal carcinoma in situ but not in normal or uninvolved epithelial cells. Positive-staining tumor epithelial cells were detected in metastases to the lymph node. HERV-K env protein expression in ovarian cancer, benign ovarian cyst, and adjacent uninvolved (normal) biopsies by immunohistochemistry. Immunohistochemistry staining was done for the HERV-K env protein in endometrioid adenocarcinoma (n=36), serous adenocarcinoma (e-h; n=226), and benign cyst (i-j) and normal epithelium biopsies (n=58 for benign and normal). The immunohistochemistry was performed using the 6H5 mAb (panels b, d, f, h, and i-I) or the isotype control A. Note positive HERV-K env staining in endometrioid adenocarcinoma (and serous adenocarcinoma (f and h) but not in benign cyst or normal ovarian epithelium. Primary melanoma tumor cells (×200) had varying staining intensity (scored 0-3) of HERV-K env expression (top) when compared with isotype IgG2a control staining (bottom). Tumor cells (×400) showed HERV-K env expression on cell membrane (punctate pattern; solid arrow) or cytoplasmic staining. Strong expression of HERV-K was detected by immunohistochemistry in most pancreatic cancer tissues containing poorly differentiated adenocarcinoma. HERV-K was not expressed in normal or matched uninvolved, non-neoplastic pancreatic tissues. The expression of HERV-K was compared in a patient with moderate differentiated adenocarcinoma including tumor biopsy and matched non-neoplastic pancreatic tissues.

### Example 5

### Anti-HERV Antibody Conjugation to Gold Nanoparticles (AuNP)

The inventors determined the isoelectric point of 6H5 mAb by isoelectric focusing. One mg of 6H5 was dialyzed against distilled water, pH 7.4, overnight at 4° C. 500 μg of dialyzed 6H5 was incubated with 50 ml of 5-nm or 30-nm colloidal gold (Ted Pella, Redding, Calif., USA) at pH 7.4 with gentle agitation for fifteen minutes at room temperature. The conjugate was loaded into a 30-ml dialysis cassette (Slide-A-Lyzer; Pierce, Rockford, Ill., USA), wrapped in cheesecloth, and left buried in drying resin (Silica gel Rubin; Sigma-Aldrich) overnight. This pre-concentration process was repeated until the sample volume was approximately three ml. A 30-ml column of Sephacryl S-500-HR (GE Healthcare) was prepared, and unincorporated label was removed by size-exclusion chromatography using ÄKTA FPLC. Fractions containing mAb-AuNP conjugate as determined by online A280 monitoring are pooled, stabilized with 0.1% polyethylene glycol 1000, and concentrated using 3 kDa molecular weight cut off centrifugal concentrators (Amicon Ultra; Millipore, Billerica, Mass., USA).

### Example 6

### Isopycnic (Density Gradient) Fractionation of HERV Virus

To identify HERV-K viral components in human plasma, heparinated blood was overlaid on Histopaque 1077 (Sigma, St. Louis, Mo., USA) density gradient media and centrifuged at 400×g for thirty minutes at room temperature. The plasma layer was retrieved and ten ml are used for isopynic separation of viral particles. Isolation of viral particles was carried out using OptiPrep iodixanol (AxisShield, Norton, Mass., USA) density gradient medium by diluting ten ml of plasma into twenty-eight ml total volume with phosphate-buffered saline. This sample was aliquoted into a polyallomer ultracentrifuge tube and 4 ml of a 50% solution of iodixanol in phosphate-buffered saline was underlaid. This sample was centrifuged at 111,800×g (average) for ninety minutes in an AH629 swinging bucket rotor in a Sorvall UltraPro 80 ultracentrifuge. After this pre-concentration step, the top 22-ml of media was discarded and the remaining sample was mixed with the underlying iodixanol cushion to give a final sample concentration of approximately 20% iodixanol. The samples are the loaded into crimp-top polyallomer tubes at centrifuged for five hours at 314,543×g (average) in a T865.1 fixed-angle rotor. Samples are carefully removed from the rotor to avoid disturbing the gradient and each tube was punctured at the top and bottom. 200 μL fractions are drained from the bottom of the tube in order of decreasing density and collected into Eppendorf tubes. The remainder of the sample in the tube was collected into two ml fractions also in order of decreasing density and archived. Viral particles isolated by isopycnic fractionation are aliquoted and kept at −20° C. for further analysis.

Isopycnic fractionation of viral particles from conditioned cell culture media from MCF-7 was carried out in the same way as plasma, only twenty-eight ml of undiluted media was used in lieu of diluted plasma and six fraction preparations are pooled and analyzed.

### Example 7

### Patient Tissue HERV Viral Particle Isolation

100 mg of snap-frozen patient tissue was homogenized using a conical mortar and pestle in one ml of RPMI-1640+10% FBS. This triturate was then passed through a 0.45μ SFCA filter, aliquoted, and frozen at −80° C. for future analysis. These samples were used only in RT-PCR experiments.

### Example 8

### HERV Reverse Transcriptase Activity Assay

Patient plasma density gradient fractions are thawed and mixed thoroughly. 50 μL was removed and dissociated with 10 mM Tris base pH 8.0+0.2 mM DTT+0.2% Igepal CA-630 with constant agitation at room temperature for one hour. Dissociated viral particles are assayed for reverse transcriptase activity using EnzChek RT Assay (Invitrogen, Carlsbad, Calif., USA) as per manufacturer's instructions. This assay uses reverse transcriptase enzyme from the sample to carry out reverse transcription on a pre-annealed poly (A) primer and oligo dT template. The resulting RNA:DNA heteroduplexes are stained with PicoGreen and the plate is read in fluorescence mode with a Victor V plate reader (Perkin Elmer, Waltham, Mass., USA).

### Example 9

### RT-PCR Assay of HERV RNA Expression in Biological Samples

Viral RNA was purified from fifty μL of each assayed gradient fraction using the MinElute Viral RNA purification kit (Qiagen, Valencia, Calif., USA). vRNA was eluted in 50 μL of buffer AVE; 5 μL of this eluate was used for RT-PCR using K10 (type 1) and K22 (type 2) primers. Total RNA from the HERV-K+ malignant breast adenocarcinoma cell line Sk-Br-3 (ATCC, Manassas, Va., USA) was used for K10 and K22 positive control. Template-free negative controls are included as well to assay for contamination of reaction components. An aliquot of each viral RNA was also treated with 0.1 mg/ml RNase A for one hour at 37° C. and setup as a parallel reaction with K10 primers to determine the contribution of genomic DNA contamination to the PCR result. Subsequent RT-PCR was carried out using the OneStep RT-PCR kit (Qiagen, Valencia, Calif., USA) to amplify Type 1 and Type 2 HERV-K Env SU vRNAs using K10 sense (5′-AGAAAAGGGCCTCCACGGAGATG-3) (SEQ ID NO: 3), K22 sense (5′-GTATGCTGCTTGCAGCCTTGATGAT-3′) (SEQ ID NO: 4), and K10 antisense (3′-ACTGCAATTAAAGTAAAAATGAA-5) (SEQ ID NO: 5) primers. Type 1 and type 2 HERV-K amplicons are both generated using the same antisense primer. 5 μL of each vRNA was used as the template in each reaction with a final reaction volume of 18 μL. Hot start PCR was carried out on a DNA Engine (PTC-200, Bio-Rad, Irvine, Calif., USA) thermocycler using the following program: 50° C. for 30 minutes (reverse transcription reaction), 95° C. for fifteen minutes (required for activation of HotStart Taq polymerase), then thirty-five cycles of thirty seconds at 94° C., thirty seconds at 55° C., and one minute at 72° C. A final extension for ten minutes at 72° C. completed the RT-PCR. RT-PCR reactions are resolved by 1% agarose gel electrophoresis in TAE (Tris, acetate, EDTA) buffer; once the reaction was complete, 6× gel loading buffer was added directly to the reaction, the entire sample was loaded, and electrophoresed for 75 Vhr. Gels are stained in TAE buffer containing 0.5 μg/ml ethidium bromide for one hour with gentle agitation and imaged on a Gel Doc (Gel Doc 1000, Bio-Rad, Irvine, Calif., USA) gel documentation system using UVB transillumination.

### Example 10

### HERV RNA Copy Number Assay of RNA Expression in Biological Samples

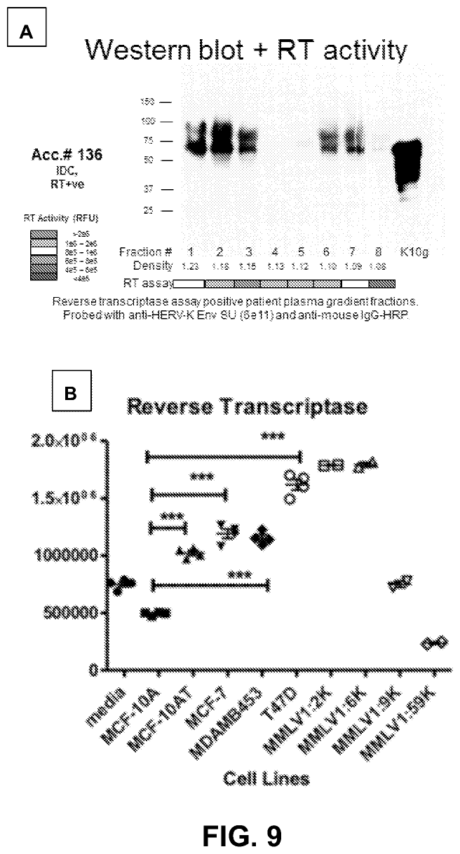

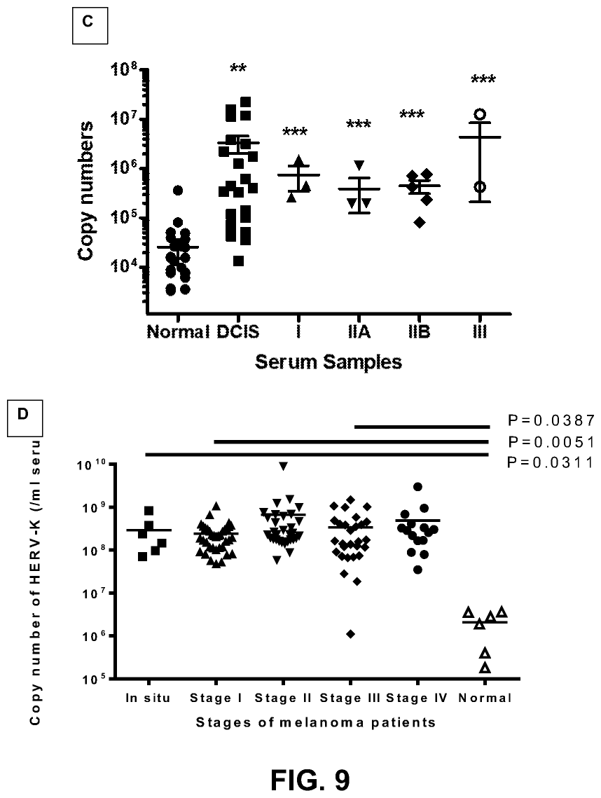

Viral RNAs (vRNAs) (60 μl) are isolated from 140 μl of serum using the QIAamp Viral RNA Mini Kit (Qiagen, Valencia, Calif., USA), following the manufacturer's instructions. Quantitative real-time RT-PCR (qRT-PCR) is carried out using the Taq Man® OneStep RT-PCR Master Mix Reagents Kit (Applied Biosystems, Foster City Calif., USA). The vRNA abundance was calculated in a ‘copy-per-ml’ format, based on a standard curve generated from serial dilution of cRNA of HERV-K genes See FIG. 9 for assays of breast cancer and melanoma patients and normal controls.

### Example 11

### 1-D SDS-PAGE and Western Blot

For HERV-K10, a 10% acrylamide denaturing SDS-PAGE was carried out. 10 μg of purified protein was heated in reducing Laemmli sample buffer at 95° C. for ten minutes before loading onto the gel. For 6H5 and 6E11, 10 μg of purified antibody are heated in non-reducing Laemmli sample buffer for five minutes before loading onto a 4% SDS-PAGE gel. A pre-stained molecular weight reference marker was included on all SDS-PAGE experiments. Gels are electrophoresed in Laemmli running buffer at 100 Vhr until the bromophenol blue dye front cleared the bottom of the gel. At this point, gels bound for Coomassie stain are removed and fixed in destain solution (10% acetic acid, 40% dH2O, and 50% methanol) for 30 minutes. The gels are then stained for ten minutes in a Coomassie R250 solution (50% methanol, 40% dH2O, 10% acetic acid+0.25% Coomassie R250) that was preheated to 65° C. and subsequently bathed in destain solution until protein bands become clearly visible and the level of background stain is acceptable. These Coomassie-stained gels are dried onto filter paper using a SGD 4050 gel dryer (Savant, East Lyme, Conn., USA) at 65° C. for three hours. Samples bound for Western blotting are removed, sandwiched in the transfer cassette of the Mini-Protean 2 wet blotting apparatus (Bio-Rad, Irvine, Calif., USA) between filter paper and adjacent a piece of 0.2μ PVDF membrane prewetted with pure methanol and equilibrated in transfer buffer. The transfer was carried out at 60 mA overnight at 4° C. The next day the membranes are removed, blocked in phosphate-buffered saline+0.2% Tween 20+3% BSA for one hour at room temperature with gentle agitation, and probed with the indicated antibodies.

For Western blot of total cell lysates, cells were cultured to 95% confluence in the media formulation recommended by ATCC. Cells were washed in phosphate-buffered saline, resuspended in 1% CHAPS+protease inhibitor cocktail (Roche, Indianapolis, Ind., USA) and frozen overnight at −80° C. Total protein concentration of clarified lysates was determined by Bradford assay and 50 μg of lysate are boiled in Laemmli sample buffer and resolved by 10% SDS-PAGE along with a lane of Kaleidoscope Precision Plus ladder (BioRad, Hercules, Calif., USA). PVDF was pre-wetted in methanol and equilibrated in Laemmli transfer buffer; samples separated on the SDS-PAGE gel are wet-transferred onto this conditioned PVDF membrane overnight at 60 mA at 4° C. The next day membranes are blocked with 3% BSA in Tris-buffered saline (TBS)+0.2% Tween-20 for one hour at room temperature with gentle agitation. 1 μg/ml 6H5 was added for one hour in blocking buffer and rinsed thoroughly in TBS-0.2% Tween-20. 1 μg/ml anti-mouse IgG-HRP conjugate was added for one hour at room temp in blocking buffer and washed thoroughly in wash buffer, followed by Tris-buffered saline only. ECL reagent (Western Lightening Plus, Perkin Elmer, Waltham, Mass., USA) was added to the membrane and exposed for various amounts of time to Biomax light ECL imaging film (Kodak, Rochester, N.Y., USA). Films are developed using standard developer and fixer chemistry (Merry Xray, San Antonio, Tex., USA).

Patient plasma fractions are prepared for Western blotting by chloroform:methanol precipitation. To 25 μL of each plasma fraction, 100 μL of methanol was added and the sample was vortexed for five seconds. 25 μL of chloroform was then added and vortexed again. 75 μL of dH2O was then added and vortexed. The sample was then centrifuged at 15,000×g for two minutes and the supernatant discarded. 100 μL of methanol was added to the pellet, vortexed again, and centrifuged at 15,000×g for two minutes. The supernatant was discarded and the remaining solvent was purged from the sample by drying the pellet in a SpeedVac (Savant, East Lyme, Conn., USA) for ten minutes. The pellet was dissolved in Laemmli sample buffer modified with 6 M urea, 1% CHAPS, and 50 mM DTT. Samples are resolved by 10% SDS-PAGE, transferred onto PVDF membranes, and blocked with 5% non-fat milk in TBS+0.2% Tween-20 (BLOTTO). The blocked membranes are probed with 10 μg/ml 6E11 for one hour at room temperature, washed in TBS+0.2% Tween-20, and probed with 10 μg/ml anti-mouse IgG-HRP. After thorough washing in TBS-T, the membranes are rinsed in TBS, and ECL reagent (Western Lightening Plus, Perkin Elmer, Waltham, Mass., USA) was added to the membrane and exposed for various amounts of time to Biomax light ECL imaging film (Kodak, Rochester, N.Y., USA). Films are developed using standard developer and fixer chemistry (Merry Xray, San Antonio, Tex., USA).

### Example 12

### Patient Plasma Gradient Fraction Analysis

Reverse transcriptase enzyme activity in a diverse selection of patient plasma and normal donor density gradient fractions was assayed using the EnzChek RT kit (Invitrogen, Carlsbad, Calif., USA), which uses a non-radioactive reporter and relies on reverse transcriptase from the assay sample generating RNA:DNA duplexes using a provided poly (A) RNA template pre-annealed to an oligo dT primer. Dissociated viral particles isolated from plasma fractions provide reverse transcriptase in the assay and the resulting RNA-DNA heteroduplexes formed by reverse transcriptase enzyme activity are stained and measured by a fluorescent stain that is highly specific for duplex nucleic acids. Samples with greater reverse transcriptase enzyme activity correspond to higher concentrations of nucleic acid heteroduplex which give higher relative fluorescence unit (RFU) readings. Each EnzChek RT assay batch included a Moloney Murine Leukemia Virus Reverse Transcriptase (MMLV-RT) enzyme standard curve in dilutions that spanned the dynamic range of the assay and gave a convenient way to correlate reverse transcriptase activity detected in patient samples to a defined number of reverse transcriptase enzyme units per unit of sample.

TABLES 1-5 show the disease status and pool reverse transcriptase enzyme activity values for breast cancer, ovarian cancer and benign disease, and healthy normal donors, respectively.

TABLE 1
Patient plasma fraction RT activity by pool and disease status-DCIS
Gradient Fraction Pool Reverse Transcriptase Value

| Acc. # | A | B | C | D | Disease | +ve? |
|---|---|---|---|---|---|---|
| 120 | 3146946 | 1234403 | 590791 | 391091 | DCIS | + |
| 127 | 1690669 | 940418 | 613821 | 535262 | DCIS | + |
| 173 | 959453 | 570692 | 405079 | 334462 | DCIS | + |
| 171 | 892942 | 1237300 | 593728 | 461829 | DCIS | + |
| 39 | 795948 | 819758 | 533416 | 467753 | DCIS | + |
| 157 | 687890 | 762709 | 448197 | 386531 | DCIS | + |
| 50 | 615483 | 533322 | 437016 | 398782 | DCIS | |
| 99 | 565128 | 395387 | 313325 | 267588 | DCIS | |
| 35 | 534324 | 514503 | 429078 | 391293 | DCIS | |
| 131 | 440039 | 410624 | 304537 | 283905 | DCIS | |
| 79 | 374216 | 681383 | 1106323 | 306653 | DCIS | + |
| 86 | 293832 | 307818 | 265460 | 227706 | DCIS | |
| 83 | 254463 | 261963 | 192109 | 176000 | DCIS | |
| | | | | | total DCIS | 13 |
| | | | | | # DCIS | 7 |
| | | | | | positive | |
| | | | | | % DCIS | 53.8 |
| | | | | | positive | |

TABLE 2
Patient plasma fraction RT activity by pool and disease
status-invasive breast cancer
Gradient Fraction Pool Reverse Transcriptase Value

| Acc. # | A | B | C | D | Disease | +ve? |
|---|---|---|---|---|---|---|
| 108 | 1663377 | 698919 | 362856 | 271631 | IDC | + |
| 147 | 1444931 | 630595 | 473537 | 390047 | IDC | + |
| 109 | 1029950 | 591706 | 579065 | 443621 | IDC | + |
| 128 | 914577 | 532992 | 416109 | 368317 | IDC | + |
| 114 | 791008 | 555204 | 376870 | 306360 | IDC | + |
| 110 | 767672 | 360612 | 269517 | 236009 | IDC | + |
| 163 | 749409 | 1582853 | 1150894 | 832558 | IDC | + |
| 45 | 680608 | 510478 | 416009 | 384297 | IDC | |
| 55 | 677296 | 454960 | 402580 | 370610 | DCIS + IDC | |
| 133 | 616209 | 681671 | 414601 | 321800 | IDC | |
| 28 | 605161 | 649789 | 473314 | 407155 | IDC | |
| 52 | 591888 | 469422 | 398436 | 370830 | IDC | |
| 73 | 546438 | 298224 | 223078 | 194778 | IDC | |
| 36 | 542724 | 592988 | 454264 | 411278 | IDC | |
| 155 | 528752 | 1008470 | 473398 | 352257 | IDC | + |
| 76 | 525870 | 382446 | 321858 | 304557 | ILC | |
| 2 | 524923 | 361397 | 320211 | 290039 | IDC | |
| 12 | 477174 | 396590 | 343956 | 302052 | IDC | |
| 48 | 180754 | 154723 | 145897 | 137889 | DCIS + tubular | |
| | | | | | carcinoma | |
| | | | | | total invasive | 19 |
| | | | | | cancer | |
| | | | | | # invasive | 8 |
| | | | | | cancer positive | |
| | | | | | % invasive | 42.1 |
| | | | | | cancer positive | |

TABLE 3
Patient plasma fraction RT activity by pool and disease
status-ovarian cancer
Gradient Fraction Pool

| Acc. | | | | | | |
|---|---|---|---|---|---|---|
| # | A | B | C | D | Disease | +ve? |
| 112 | 1628379 | 1438226 | 1113414 | 709821 | adenocarcinoma, | + |
| | | | | | IIIc | |
| 204 | 1203391 | 910734 | 564569 | 358905 | serous | + |
| | | | | | adenocarcinoma | |
| 174 | 1038934 | 545113 | 338341 | 437531 | GI stromal tumor | + |
| | | | | | metastatic | + |
| 65 | 941650 | 784501 | 688987 | 536367 | adenocarcinoma | |
| 141 | 701056 | 568797 | 436871 | 358138 | endometrial | + |
| | | | | | adenocarcinoma | |
| 158 | 659482 | 696844 | 664830 | 513360 | high grade ovarian | |
| | | | | | carcinoma | |
| 105 | 598699 | 770691 | 421033 | 314240 | Mullerian tumor | + |
| 67 | 565812 | 1291063 | 691391 | 593853 | high-grade serous | + |
| | | | | | adenocarcinoma | |
| 125 | 531077 | 521725 | 364645 | 304045 | LMP ovarian tumor | |
| 115 | 483775 | 695919 | 1643799 | 1880087 | malignant mixed | + |
| | | | | | Mullerian tumor | |
| 153 | 478361 | 428438 | 350288 | 312882 | papillary serous | |
| 92 | 468215 | 1140544 | 1546342 | 936441 | papillary serous | + |
| | | | | | carcinoma | |
| | | | | | total ovarian cancer | 12 |
| | | | | | # ovarian cancer | 9 |
| | | | | | positive | |
| | | | | | % ovarian cancer | 75 |
| | | | | | positive | |

TABLE 4
Patient plasma fraction RT activity by pool and disease
status-ovarian benign
Gradient Fraction Pool

| Acc. | | | | | | |
|---|---|---|---|---|---|---|
| # | A | B | C | D | Disease | +ve? |
| 202 | 698314 | 503500 | 339894 | 290460 | benign control | |
| 206 | 685244 | 408884 | 297672 | 260292 | benign cyst | |
| 169 | 643008 | 609756 | 446303 | 319128 | benign cyst teratoma | |
| 164 | 602892 | 1094879 | 631250 | 458842 | ovarian cyst | + |
| 186 | 579883 | 360603 | 290234 | 253426 | benign teratoma | |
| 165 | 517323 | 386792 | 297022 | 255242 | benign control | |
| 142 | 511385 | 467847 | 421010 | 366963 | benign | |
| | | | | | cystadenofibroma | |
| 101 | 494751 | 476209 | 323066 | 284746 | teratoma control | |
| 71 | 426943 | 389099 | 295179 | 258782 | benign control | |
| | | | | | total ovarian benign | 9 |
| | | | | | # ovarian positive | 1 |
| | | | | | % ovarian positive | 11.1 |

TABLE 5
Patient plasma fraction RT activity by pool and
disease status-normal
Gradient Fraction Pool RT Value

| Acc. # | A | B | C | D | Disease | RT + ve? |
|---|---|---|---|---|---|---|
| ND 251040 | 348246 | 285439 | 204213 | 181054 | − | |
| ND 273010 | 376103 | 318805 | 279443 | 250364 | − | |
| ND 328706 | 440958 | 306814 | 222697 | 165254 | − | |
| ND 329966 | 310911 | 884544 | 580280 | 313170 | − | + |
| ND 452635 | 641884 | 539045 | 361070 | 317444 | − | |
| ND 452636 | 286062 | 273739 | 277796 | 210218 | − | |
| | | | | | total | 6 |
| | | | | | normal | |
| | | | | | # | 1 |
| | | | | | normal | |
| | | | | | positive | |
| | | | | | % | 16.67 |
| | | | | | normal | |
| | | | | | positive | |

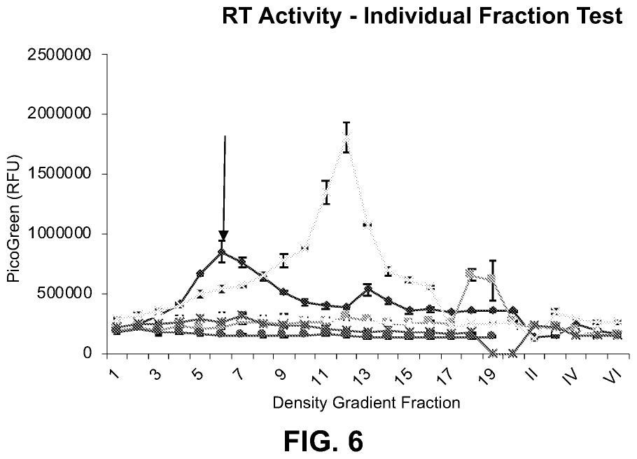

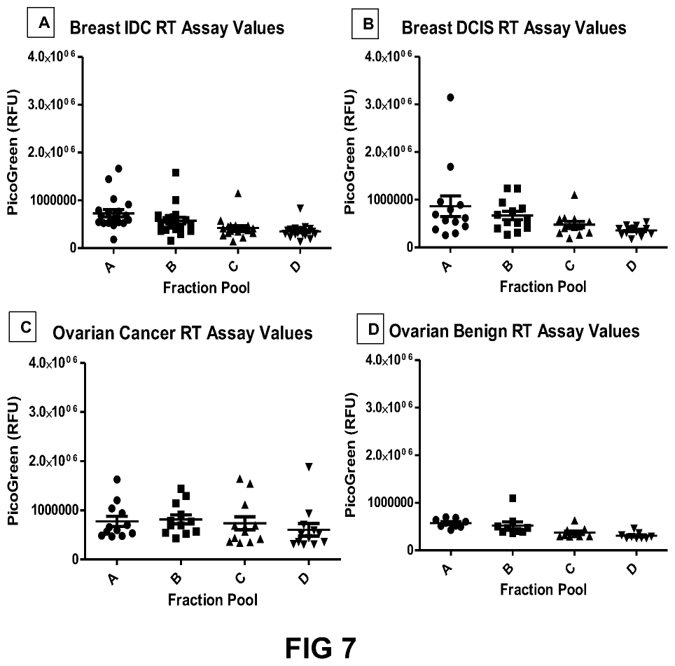

FIG. 6 show a representative line graph of individual fraction reverse transcriptase assays Fractions showing peak activity are assayed by RT-PCR. The dot plots in FIG. 7 and FIG. 8 show the distribution of plasma reverse transcriptase enzyme activity assay results by pool for breast cancer, ovarian cancer, and benign ovarian disease, respectively. FIG. 7 shows dot plots of (A) invasive breast cancer patient, (B) in situ breast cancer patient, (C) malignant ovarian disease patient, and (D) ovarian benign disease patient plasma fraction reverse transcriptase assay results by pool. FIG. 8 further shows (A) dot plot of patient plasma reverse transcriptase assay pool value t-test for DCIS patient vs. normal donor samples. dA-dD are DCIS patient pools and nA-nD are normal patient pools, (B) Patient plasma reverse transcriptase assay pool value t-test for invasive breast cancer patient vs. normal donor samples. iA-iD are invasive breast cancer patient pools and nA-nD are normal donor pools, (C) patient plasma reverse transcriptase assay pool value t-test for malignant and benign ovarian cancer patients compared to normal donor samples. oA-oD are ovarian cancer plasma pools, bA-bD are benign tumor patient pools, and nA-nD are normal donor plasma pools. Stars indicate p<0.05 comparing the groups indicated.

Individual plasma fractions from representative patients and a normal donor are selected to assay for presence of HERV-K Env surface antigen by Western blotting using anti-HERV-K monoclonal antibody 6E11 and anti-mouse IgG-HRP as the probe. The results from the reverse transcriptase activity assay and Western blot, along with the measured fraction density are compiled for these samples and shown in FIG. 9, which shows fraction density, Western blot, and reverse transcriptase activity of (A) Patient Acc. #136, (B) Reverse transcriptase activity was compared in cell culture media from various breast cell lines. Serial dilutions of murine leukemia virus reverse transcriptase (Stratagene, La Jolla, Calif., USA) are used as calibrators. Significantly greater reverse transcriptase activity (P<0.001) was demonstrated in all BC cell lines than in MCF-10A or MCF-10AT cells using one-way ANOVA assay. (C) Select fractions showing high reverse transcriptase activity are used to isolate viral RNA and perform RT-PCR using HERV-K Env type 1 and type 2 primers, to verify the expression of HERV-K in sera. The sample loading is as follows: amplicon from type 1 primers without RNase treatment, amplicons from type 1 primers with RNase treatment, and amplicons from type 2 primers without RNase treatment. Since the vRNA template is inside of the virus, as well as the presence of heparin which interferes with downstream RT-PCR of the plasma fractions, samples are processed with Qiagen's MinElute Viral RNA kit. A sample of these RT-PCR amplicons was digested with RNase A as a control. The amplicons are resolved on 1% agarose-TAE gels, stained with ethidium bromide, and imaged. Increased expression of HERV-K viral mRNA was observed in BC patient samples with accession (Acc.) #155 and Acc. #73. No expression of HERV-K viral mRNAs was detected in a patient sample (Acc. #163), who had a low HERV-K copy number by quantitative reverse transcriptase-polymerase chain reaction (qRT-PCR). (D) shows viral load (HERV-K mRNA copy number), which was determined in RNA obtained from patient sera by qRT-PCR. A higher viral load was found in all breast cancer sera, especially in ductal carcinoma in situ (DCIS), compared with normal female donors, and in all melanoma patients, compared with normal donors.

From the samples that yielded an amplicon, specific bands are excised, purified, and submitted for sequencing with an HERV-K Env SU-specific primer. The reported sequences are processed by NCBI's BLAST algorithm and the results of the highest scoring alignments are shown in TABLE 6.

TABLE 6
Top-scoring BLAST alignments from sequenced RT-PCR amplicons

| | Top Scoring BLAST | | | |
|---|---|---|---|---|
| Patient #/Disease Status | Alignment | Score | E value | Acc. Number |
| Acc. # 104 = serous/high- | Human endogenous | 1044 | 0.0 | AY037929.1 |
| grade serous carcinoma | retrovirus K115 | | | |
| (ovarian cancer) | | | | |
| Acc. # 163 = invasive | Homo sapiens isolate | 1450 | 0.0 | DQ069912.1 |
| ductal carcinoma (IDC, | T842 endogenous virus | | | |
| breast cancer) | HERV-K envelope | | | |
| Acc. # 155 = invasive | Human endogenous | 953 | 0.0 | EU308984.1 |
| ductal carcinoma | retrovirus K isolate | | | |
| (breast cancer) | HD8.13 | | | |
| Acc. # 127 = ductal | Homo sapiens isolate | 126 | 4e−26 | JN675021.1 |
| carcinoma in situ (DCIS, | HML-2_3q12.3 | | | |
| breast cancer) with | endogenous virus | | | |
| RNase treatment | HERV-K | | | |
| Acc. # 127 = DCIS | Human endogenous | 174 | 2e−40 | EU308977.1 |
| | retrovirus K isolate | | | |
| | HD8.5 | | | |
| Acc. # 115 = malignant | Homo sapiens isolate | 857 | 0.0 | DQ069912.1 |
| mixed Mullerian tumor | T842 endogenous virus | | | |
| (MMMT, ovarian cancer) | HERV-K envelope | | | |
| peak 1 | | | | |
| Acc. # 115 = MMMT | Homo sapiens | 1426 | 0.0 | JN656291.1 |
| (ovarian cancer) peak 2 | endogenous virus | | | |
| | HERV-K chromosome 19 | | | |
| Acc. # 104 = serous/high- | Human endogenous | 699 | 0.0 | DQ360575.1 |
| grade serous carcinoma | retrovirus K clone 5b8 | | | |
| (ovarian cancer) | | | | |
| Sk-Br-3 (ATCC malignant | Homo sapiens isolate | 695 | 0.0 | JN675080.1 |
| breast cancer cell line, | HML-2_19q11 | | | |
| positive control) | endogenous virus | | | |
| | HERV-K | | | |

### Example 13

### Lateral Flow Immunoassay of HERV Antibody or Protein.

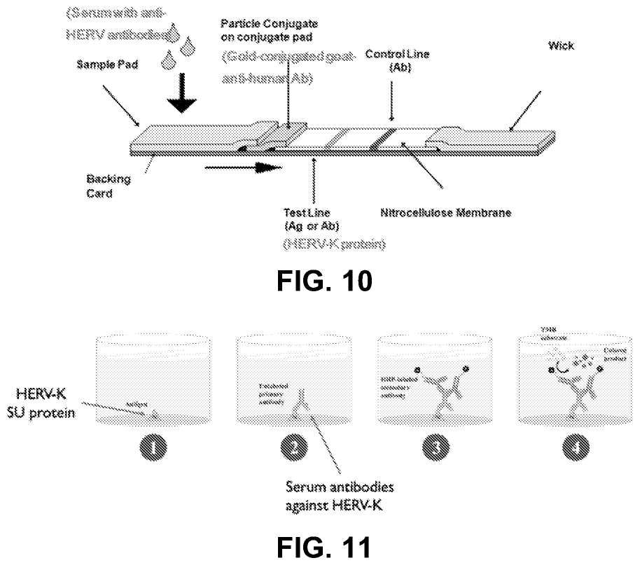

In the results shown in FIG. 10, colloidal gold (40-nm)-conjugated goat anti-human IgG is sprayed onto the conjugate pad, and recombinant HERV protein antigen is striped in a test line. A goat anti-human IgG is striped in the control line position. Colloidal gold enjoys widespread use as a label for antibody in home test strips, because the pink color formed as a result of an immunoreaction between antigen and the colloidal gold-labeled antibody is easy to recognize visually without an instrument. The inventors regularly conjugated antibodies to gold. The addition of a chase buffer causes the conjugate and sample to migrate across the test strip, sequentially contacting the HERV test line and the control line, which binds the human IgG present in the sample. The anti-human IgG-gold conjugate migrates through the membrane in the aqueous phase until it is bound by human IgG that is present on the HERV test line and the control line. The accumulation of antibody-gold complexes captured by the test and control lines causes pink lines to form, and the color formation is complete after fifteen to twenty minutes. The formation of control and test lines indicates an HERV positive result, while the formation of a control line only indicates an HERV-negative result, and the absence of a control line is indicative of no sample being added to the assay.

An advantage of this approach is the variety of recombinant antigens and mAbs and scFvs against the HERV and other HERV proteins. The inventors optimized protein labeling and detection using an AKTA protein purification system.

Test lines are evaluated semiquantitatively by using a colorimetric scale of lines of colloidal gold striped on a card at various concentrations. A series of pink lines that develop after spraying with a colloidal gold solution, ranging in OD values from solutions that give the maximum line intensity to those giving the weakest line intensity that still can be detected, is striped on strips that are used to evaluate the intensity of test and control lines. HERV protein cutoff calibrators are used to set the visual limit of detection. These test strip values are compared to values for the same samples run using a standard ELISA assay, and ELISA cutoff calibrators are also set.

### Example 14

### Standard Curve

This EXAMPLE supports a determination of substantial equivalence for a medical device.

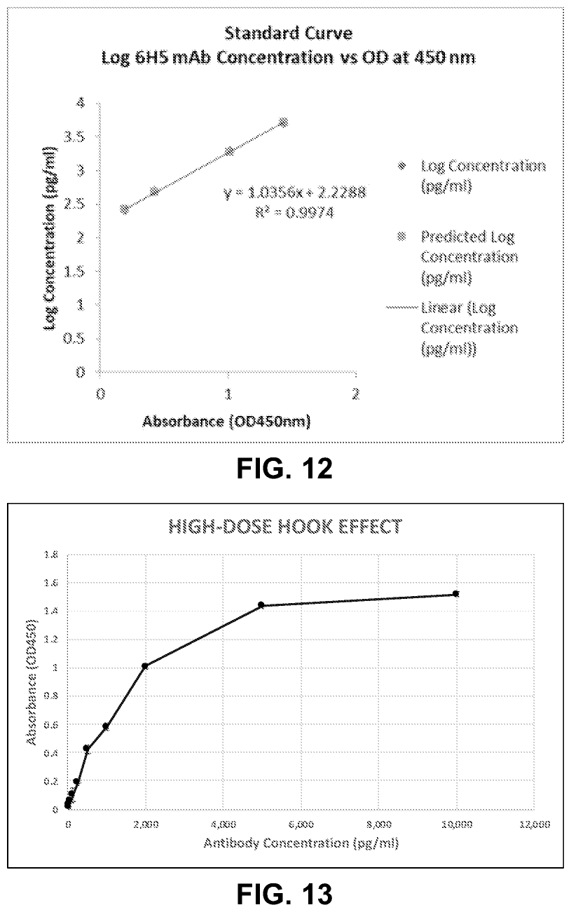

The inventors derived the standard curve shown in FIG. 12 by plotting the absorbance of the monoclonal antibody (mAb) 6H5, which has a known protein concentration, against the mAb concentration. The standard curve can be used to convert absorbance values of the assay to log of serum antibody concentration units in picograms per ml. The standard curve includes two antibody concentrations that give absorbance values in the low range of the serum samples the inventors analyzed and two concentrations that give high absorbance values.

Raw optical-density readings increase the between-run variability due to minor variations in reagents or environmental conditions. the inventors used a mAb control standard curve for each assay. This provides a precise measurement by controlling for interexperiment variability. Thus, relatively minor experimental errors in optical density values translate to large errors in calculated antibody concentrations, because the relationship between optical density and antibody concentration is log-linear. See N. Engl. J. Med., 383, 1694-1698 (2020). These errors can be controlled with an internal standard control antibody, such as 6H5 in this case. The inventors used this standard curve to convert their absorbance readings to antibody protein concentrations for the assays in their recent FDA 510(k) submission.

TABLET
Standard curve

| Concentration (pg/ml) | 250 | 500 | 2000 | 5000 |
|---|---|---|---|---|
| Log concentration | 2.39794 | 2.69897 | 3.30103 | 3.69897 |
| Absorbance at 450 nm | 0.1925 | 0.428 | 1.012 | 1.44 |

### Example 15

### Precision/Repeatability and Reproducibility

This EXAMPLE supports a determination of substantial equivalence for a medical device.

The inventors assayed in duplicate four breast cancer patient serum samples covering the assay range, two runs per day for a period of twenty days, with two reagent lots. A total of eighty values were generated per sample.

The repeatability and reproducibility are summarized in TABLE 8. The mean log of antibody concentration is given for each patient sample.

TABLE 8
Precision/repeatability and reproducibility

| | | Re- | Re- | With- | With- | Re- | Re- |
|---|---|---|---|---|---|---|---|
| Patient | | peat- | peat- | in | in | produci- | produci- |
| Sam- | | ability | ability | Day | Day | bility | bility |
| ple | Mean | SD | CV | SD | CV | SD | CV |
| 1 | 3.29 | 0.10 | 2.99% | 0.17 | 5.31% | 0.22 | 6.63% |
| 2 | 3.25 | 0.11 | 3.25% | 0.16 | 4.91% | 0.187 | 5.76% |
| 3 | 2.51 | 0.05 | 2.06% | 0.07 | 2.77% | 0.12 | 4.93% |
| 4 | 2.79 | 0.09 | 3.20% | 0.14 | 5.05% | 0.21 | 7.41% |

### Example 16

### Precision, Site-to-Site

This EXAMPLE supports a determination of substantial equivalence for a medical device.

The inventors carried out site-to-site precision and reproducibility assays at two sites. Four patients were evaluated at each laboratory sites. There were four runs per day for each patient for five days at each site. A breakdown of coefficients of variation for each of the four patients is in TABLE 9.

TABLE 9
Precision, site-to-site

| | | | | Within | Within | Re- | Re- |
|---|---|---|---|---|---|---|---|
| | | Repeat- | Repeat- | Labora- | Labora- | produci- | produci- |
| Patient | | ability | ability | tory | tory | bility | bility |
| Sample | Mean | SD | CV | SD | CV | SD | CV |
| 1 | 3.26 | 0.15 | 4.72% | 0.20 | 6.01% | 0.21 | 6.32% |
| 2 | 3.23 | 0.16 | 4.97% | 0.17 | 5.38% | 0.17 | 5.42% |
| 3 | 2.51 | 0.06 | 2.42% | 0.08 | 3.09% | 0.14 | 5.64% |
| 4 | 2.78 | 0.14 | 5.01% | 0.18 | 6.64% | 0.18 | 6.64% |

### Example 17

### Precision, Lot-to-Lot

This EXAMPLE supports a determination of substantial equivalence for a medical device.

A lot-to-lot assay precision assay was conducted to demonstrate the reproducibility of anti-HERV-K serum antibody assays using two lots of HERV-K antigen across collection sites and analyzed by the same operator, in accordance with CLSI Guideline EP05-A3. The Precision results and a breakdown of coefficients of variation for each of the four patients in each lot are presented below.

TABLE 10
Precision, lot-to-lot

| | | | | Within | Within | Re- | Re- |
|---|---|---|---|---|---|---|---|
| | | Repeat- | Repeat- | Labora- | Labora- | produci- | produci- |
| Patient | | ability | ability | tory | tory | bility | bility |
| Sample | Mean | SD | CV | SD | CV | SD | CV |
| 1 | 3.24 | 0.13 | 3.93% | 0.23 | 6.95% | 0.24 | 7.36% |
| 2 | 3.20 | 0.12 | 3.86% | 0.18 | 5.56% | 0.18 | 5.56% |
| 3 | 2.56 | 0.08 | 3.10% | 0.11 | 4.26% | 0.12 | 4.58% |
| 4 | 2.76 | 0.13 | 4.62% | 0.22 | 7.80% | 0.22 | 7.80% |

### Example 18

### Precision, Investigator-to-Investigator

This EXAMPLE supports a determination of substantial equivalence for a medical device.

The inventors conducted an inter-investigator precision study to demonstrate the reproducibility of analyzing patient serum samples for anti-HERV-K antibody levels by different operators across collection sites using the same lot of manufactured HERV-K antigen, in accordance with CLSI Guideline EP05-A3. Inter-investigator antibody levels for each of four patients are summarized in TABLE 11.

TABLE 11
Patient #1

| | | Repestability | Repeatability | Within Day | Within Day | Reproducibility | Reproducibility |
|---|---|---|---|---|---|---|---|
| Investigator | Mean | SO | CV | SO | CV | SD | CV |
| 1 | 3.33313795 | 0.10126444 | 3.0% | 0.19389743 | 5.8% | 0.19389743 | 5.8% |
| 2 | 3.11494998 | 0.09697628 | 3.1% | 0.15296039 | 4.9% | 0.19248389 | 6.2% |
| 3 | 3.31546803 | 0.09084808 | 2.7% | 0.12640101 | 3.8% | 0.30326924 | 9.1% |

Patient #2

| | | Repeatability | Repeatability | Within Day | Within Day | Reproducibility | Reproducibility |
|---|---|---|---|---|---|---|---|
| Investigator | Mean | SD | CV | SD | CV | SD | CV |
| 1 | 2.911638649 | 0.043393019 | 1.7% | 0.052440794 | 2.1% | 0.117356768 | 4.7% |
| 2 | 2.476632025 | 0.084145686 | 3.4% | 0.108226394 | 4.4% | 0.127865631 | 5.2% |
| 3 | 2.536198443 | 0.024155647 | 1.0% | 0.065372065 | 2.6% | 0.157620305 | 6.2% |

Patient #3

| | | Repeatability | Repeatsblity | Within Day | Within Day | Reproducibility | Reproducibility |
|---|---|---|---|---|---|---|---|
| Investigator | Mean | SD | CV | SD | CV | SO | CV |
| | 2.81574788 | 0.08174751 | 2.9% | 0.12382441 | 4.4% | 0.17646150 | 6.3% |
| 2 | 2.72860645 | 0.04912828 | 1.8% | 0.20174889 | 7.4% | 0.21019717 | 7.7% |
| 3 | 2.76058060 | 0.13155942 | 4.8% | 0.13155942 | 4.8% | 0.31418872 | 11.4% |

Patient #4

| | | Repeatability | Repeatability | Within Day | Within Day | Reproducibility | Reproducibility |
|---|---|---|---|---|---|---|---|
| Investigator | Mean | SD | CV | SO | CV | SD | CV |
| 1 | 3.26096958 | 0.11111255 | 3.4% | 0.15415410 | 4.7% | 0.19777686 | 6.1% |
| 2 | 3.25469125 | 0.12921885 | 4.0% | 0.21302310 | 6.5% | 0.21302310 | 6.5% |
| 3 | 3.22873653 | 0.04625479 | 1.4% | 0.10475057 | 8.2% | 0.17113179 | 5.3% |

### Example 19

### Linearity/Assay Reportable Range

This EXAMPLE supports a determination of substantial equivalence for a medical device.

Two samples (patients #1 and #3) with relatively high HERV-K antibody titers and two naturally low-titer samples (patients #2 and #4) were serially diluted to 1/8 with the HERV-K antibody diluent. Linearity and recovery after dilution were studied according to a protocol based on the recommendations of the document CLSI® EP06-A, with error criteria set at 0.1 log pg/ml for repeatability. The assay was shown to be linear over the measuring range, with allowable nonlinearity within the set error criteria.

TABLE 12

| | | Predicted | Measured | |
|---|---|---|---|---|
| | | Ab Conc. | Ab Conc. | Recovery |
| Sample | Dilution | (log pg/mL) | (log pg/mL) | % |
| Patient # 1 | 1 | 3.404 | 3.435 | 100.91 |
| | ½ | 3.124 | 3.066 | 98.14 |
| | ¼ | 2.926 | 2.881 | 98.46 |
| | ⅛ | 2.717 | 2.789 | 102.65 |
| Patient #2 | 1 | 2.903 | 2.921 | 100.62 |
| | ½ | 2.692 | 2.658 | 98.74 |
| | ¼ | 2.544 | 2.526 | 99.29 |
| | ⅛ | 2.426 | 2.461 | 101.44 |
| Patient # 3 | 1 | 3.325 | 3.366 | 101.23 |
| | ½ | 3.119 | 3.047 | 97.69 |
| | ¼ | 2.960 | 2.888 | 97.57 |
| | ⅛ | 2.706 | 2.809 | 103.81 |
| Patient # 4 | 1 | 3.157 | 3.192 | 101.11 |
| | ½ | 2.944 | 2.872 | 97.55 |
| | ¼ | 2.740 | 2.712 | 98.98 |
| | ⅛ | 2.567 | 2.632 | 102.53 |

### Example 20

### Sterilization and Shelf Life

This EXAMPLE supports a determination of substantial equivalence for a medical device.

Storage of coated ELISA plates at 37° C. for 2 months did not result in a change in absorbance readings. See Table 13 and TABLE 14, An accelerated aging analysis indicates that these conditions are equivalent to a real-time shelf life of 5-6 months

TABLE 13

| | Patient | Patient | Patient | Patient | Patient |
|---|---|---|---|---|---|
| 37° C. overnight plate | #1 | #2 | #3 | #4 | #5 |
| Log Ab conc (pg/ml) | 3.829 | 3.697 | 3.391 | 3.220 | 3.724 |
| St. Dev. | 0.195 | 0.341 | 0.142 | 0.032 | 0.008 |

TABLE 14

| | Patient | Patient | Patient | Patient | Patient |
|---|---|---|---|---|---|
| 37° C. 2-month plate | #1 | #2 | #3 | #4 | #5 |
| Log Ab Conc (pg/ml) | 3.832 | 3.621 | 3.374 | 3.171 | 3.643 |
| Std. Dev. | 0.081 | 0.308 | 0.064 | 0.044 | 0.015 |

### Example 21

### High Dose Hook Effect

This EXAMPLE supports a determination of substantial equivalence for a medical device.

A serum sample with high HERV-K titer gave an absorbance reading at the saturation point. The sample was diluted 1/2, starting from a concentration of 10,000 pg/ml, down to a concentration of to 4 pg/ml with sample diluent and the data plotted to demonstrate the absorbance signal relative to the serum antibody concentration. No high dose hook effect was observed for serum antibody concentrations up to 10,000 pg/ml. See FIG. 13.

### Example 22

### Analytical Specificity

This EXAMPLE supports a determination of substantial equivalence for a medical device.

Ibuprofen, acetaminophen, gentamicin, ampicillin, cisplatin, tamoxifen, doxorubicin, and paclitaxel interferents were evaluated for interference when added to five human breast cancer serum samples containing high (patients #1 and #2), moderate (patients #3 and #5), and low (patient #4) titers of anti-HERV-K serum antibodies. Stock solutions of the interferents were spiked into the anti-HERV-K antibody-containing serum samples. Controls were sera spiked with ethanol or water in volumes equivalent to the potential interferents. Aliquots of each concentration were assayed in duplicate using a 20-minute ELISA assay. The majority of the potential interferents did not significantly influence this assay, where acceptance was defined as the mean±2.5 SD relative to the controls.

Mean values of duplicate runs

| | Patient | Patient | Patient | Patient | Patient |
|---|---|---|---|---|---|
| Drug | #1 | #2 | #3 | #4 | #5 |
| Ibuprofen | 3.217 | 3.349 | 3.139 | 2.801 | 3.075 |
| Acetaminophen | 3.238 | 3.303 | 3.146 | 2.946 | 3.073 |
| Gentamicin | 3.171 | 3.271 | 3.114 | 2.901 | 3.037 |
| Ampicillin | 3.176 | 3.337 | 3.132 | 2.912 | 3.093 |
| Cisplatin | 3.199 | 3.247 | 3.107 | 2.904 | 3.129 |
| Tamoxifen | 3.207 | 3.262 | 3.077 | 2.839 | 2.941 |
| Doxorubicin | 3.143 | 3.452 | 3.241 | 2.983 | 3.028 |
| Paclitaxel | 3.100 | 3.427 | 3.150 | 3.004 | 3.075 |
| (−) Ethanol | 3.202 | 3.318 | 3.078 | 2.805 | 3.041 |
| (−) H2O | 3.220 | 3.347 | 3.045 | 2.861 | 3.065 |

### List of Embodiments

Specific compositions and methods of endogenous retroviruses as biomarkers and biomarker panels for detection, diagnosis and prognosis of cancer were described. The scope of the invention should be defined solely by the claims. A person having ordinary skill in the art shall interpret all claim terms in the broadest possible manner consistent with the context and the spirit of the disclosure. The detailed description in this specification is illustrative and not restrictive or exhaustive. This invention is not limited to the particular methodology, protocols, and reagents described in this specification and can vary in practice. When the specification or claims recite ordered steps or functions, alternative embodiments might perform their functions in a different order or substantially concurrently. Other equivalents and modifications besides those already described are possible without departing from the inventive concepts described in this specification, as those skilled in the art recognize.

All patents and publications cited throughout this specification are incorporated by reference to disclose and describe the materials and methods used with the technologies described in this specification. The patents and publications are provided solely for their disclosure before the filing date of this specification. All statements about the patents and publications' disclosures and publication dates are from the inventors' information and belief. The inventors make no admission about the correctness of the contents or dates of these documents. Should there be a discrepancy between a date provided in this specification and the actual publication date, then the actual publication date shall control. The inventors may antedate such disclosure because of prior invention or another reason. Should there be a discrepancy between the scientific or technical teaching of a previous patent or publication and this specification, then the teaching of this specification and these claims shall control.

When the specification provides a range of values, each intervening value between the upper and lower limit of that range is within the range of values unless the context dictates otherwise.

Some further embodiments of the technology described can be defined according to the following numbered paragraphs:

39. A method of detecting cancer in a patient in need of detection, diagnosis, or prognosis of cancer, or prediction of response to cancer therapy, or predicting cancer metastasis, comprising:

(i) obtaining a biological sample from a subject suspected of suffering from cancer, and

(ii) identifying, in the biological sample, an increase in the levels of at least one, or at least two, or at least three or more of the following molecules relative to a healthy control subject: anti-human endogenous retrovirus HERV protein antibody (whether from HERV-H, HERV-E, ERV-3, HERV-W, or another HERV subtype); anti-HERV peptide antibody; anti-HERV Env surface (SU) protein antibody; anti-HERV Env surface (SU) peptide antibody; anti-HERV Env transmembrane (TM) protein antibody; anti-HERV Env transmembrane (TM) peptide antibody; anti-HERV Gag protein antibody; anti-HERV Gag peptide antibody; anti-HERV Pol protein antibody; anti-HERV Pol peptide antibody; anti-HERV NP9 protein antibody; anti-HERV NP9 peptide antibody; anti-HERV Rec protein antibody; anti-HERV Rec peptide antibody; anti-HERV multiple antigen peptide (MAP) antibody; HERV protein; HERV peptide; HERV Env surface (SU) protein; HERV Env surface (SU) peptide; HERV Env transmembrane (TM) protein; HERV Env transmembrane (TM) peptide; HERV Gag protein; HERV Gag peptide; HERV Pol protein; HERV Pol peptide; HERV NP9 protein; HERV NP9 peptide; HERV Rec protein; HERV Rec peptide; HERV RNA; HERV Env surface (SU) RNA; HERV Env transmembrane (TM) RNA; HERV Gag RNA; HERV Pol RNA HERV NP9 RNA; HERV Rec RNA; HERV DNA; HERV Env surface (SU) DNA; HERV Env transmembrane (TM) DNA; HERV Gag DNA; HERV Pol DNA; HERV NP9 DNA; HERV Rec DNA; immune checkpoint molecule PD-1 protein; immune checkpoint molecule PD-1 RNA; immune checkpoint molecule PD-L1 protein; immune checkpoint molecule PD-L1 RNA; immune checkpoint molecule PD-L2 protein; immune checkpoint molecule PD-L2 RNA; immune checkpoint molecule CTLA-4 protein; immune checkpoint molecule CTLA-4 RNA; immune checkpoint molecule LAG3 protein; immune checkpoint molecule LAG3 RNA; immune checkpoint molecule TIM3 protein; immune checkpoint molecule TIM3 RNA; immune checkpoint molecule CD27 protein; immune checkpoint molecule CD27 RNA; immune checkpoint molecule B7 family protein; immune checkpoint molecule B7 family RNA; HERV reverse transcriptase (RT) activity; a control (consensus) HERV immunosuppressive domain region (TMc) protein sequence; a control (consensus) HERV immunosuppressive domain region (TMc) RNA sequence; a control (consensus) HERV immunosuppressive domain region (TMc) protein with sequence GIHWQKNSARLWNSQSSIDQKLANQINDLRQTVIWMGDRLMSLEHRFQLQCDC (SEQ ID NO: 1); a HERV RNA sequence that codes for a control (consensus) (TMc) immunosuppressive domain region with protein sequence GIHWQKNSARLWNSQSSIDQKLANQINDLRQTVIWMGDRLMSLEHRFQLQCDC(SEQ ID NO: 1); a variant HERV immunosuppressive domain region (TMv) region protein with sequence; IGKRILQDCGIHNLVLIKNWQIKLIILDKLIWIGDRLMSLERRFQLQCDC (SEQ ID NO: 2); and an RNA sequence that codes for a variant HERV immunosuppressive domain region (TMv) region with protein sequence

(SEQ ID NO: 2)
IGKRILQDCGIHNLVLIKNWQIKLIIL
DKLIWIGDRLMSLERRFQLQCDC.

40. The method of the thirty-ninth embodiment, wherein the HERV is HERV-K.

41. The method of the thirty-ninth embodiment, wherein the biological sample is a bodily fluid.

42. The method of embodiment 3, wherein the bodily fluid comprises peripheral blood, sera, plasma, ascites, urine, cerebrospinal fluid (CSF), sputum, saliva, bone marrow, synovial fluid, aqueous humor, cerumen, bronchioalveolar lavage fluid, semen, prostatic fluid, Cowper's fluid or pre-ejaculatory fluid, sweat, fecal matter, tears, cyst fluid, pleural fluid, peritoneal fluid, pericardial fluid, lymph, chyme, chyle, bile, interstitial fluid, pus, sebum, vomit, mucosal secretion, stool water, pancreatic juice, lavage fluids from sinus cavities, bronchopulmonary aspirates, blastocyl cavity fluid, or a combination thereof.

43. The method of the thirty-ninth embodiment, further comprising determining whether the levels of at least one of the molecules are higher than a control level, wherein the control level is determined from a group of healthy subjects or subjects without cancer.

44. The method of the thirty-ninth embodiment, further comprising determining whether an increase in the levels of at least one of the molecules, relative to a healthy subject or subject without cancer, indicates the presence of cancer in the subject.

45. The method of the thirty-ninth embodiment, further comprising selecting a therapy for cancer if the levels of at least one of the molecules are higher than the levels in a healthy subject or subject without cancer.

46. The method of the thirty-ninth embodiment, comprising selecting a subject identified as having cancer for confirmatory diagnostic cancer testing.

47. The method of the thirty-ninth embodiment, further comprising administering a therapy for cancer if the levels of at least two of the molecules are higher than the levels in a healthy subject or subject without cancer.

48. The method of the thirty-ninth embodiment, further comprising a kit for detecting HERV and HERV-associated-molecule biomarkers for cancer in a patient consisting of a) antigens or antibodies immunologically specific for two or more HERV and HERV-associated-molecule biomarkers of the thirty-ninth embodiment for detecting expression levels of said HERV and HERV-associated-molecule biomarkers in a sample obtained from said patient, wherein said labeled antigens or antibodies form specific binding pairs with said HERV and HERV-associated-molecule biomarkers, and wherein said labeled HERV and HERV-associated-molecule biomarkers are suitable for flow cytometric analysis, immunohistochemical detection, or immunoblot analysis, or labeled nucleic acids or nucleic acid primers which specifically hybridize to or amplify HERV and HERV-associated-molecule biomarkers for detecting expression levels of said HERV and HERV-associated-molecule biomarkers in a sample obtained from said patient, wherein said labeled nucleic acids amplify said HERV and HERV-associated-molecule biomarker-encoding nucleic acids, and wherein said labeled nucleic acids are suitable for performance of in situ hybridization assay, hybridization assay, gel electrophoresis, RT-PCR, real time PCR, or microarray analysis, and b) instructional materials comprising ranges of HERV and HERV-associated-molecule biomarker expression levels associated with aggressive metastatic cancer and ranges of expression levels associated with non-aggressive non-metastatic cancer.

49. An assay kit for detecting, diagnosing, and following progression of cancer in a subject, comprising at least one, at least two, or at least three or more reagents sufficient for detection of the presence or absence of an anti-HERV antibody or an HERV target in a sample, for determining the activity of cancer cells toward combinations of agents selectively targeting HERV. The kit comprises a preparation of cancer cells; combinations of agents selectively targeting HERV; and one or more reagents sufficient to perform an assay selected from groups that comprise (1) cell growth or survival assays carried out under specific culture conditions, (2) the ability to express a defined biologic factor, (3) cell structure assays, or 4) differential gene expression assays. Kit components include primers, buffers, probes, antibodies (primary and secondary) for immunolabeling and signal detection to increase signal amplification and sensitivity. Secondary antibodies can be conjugated to enzymes such as horseradish peroxidase (HRP) or alkaline phosphatase (AP); or fluorescent dyes such as fluorescein isothiocyanate (FITC), rhodamine derivatives, Alexa Fluor dyes, or other molecules to be used in various applications, enzymes, e.g., polymerases, ligases, reverse transcriptase's, nucleases, etc., components for sample isolation, sample preparation, instrumentation, software, instructions for cancer detection, diagnosis, or prognosis in the subject based on the presence or absence of targets that comprise HERV antigens, HERV nucleic acids, or anti-HERV antibodies of the thirty-ninth embodiment. The instructions provide recommendations to assist a treating physician in the course of action, based on the results of the analysis, to optimize patient care.

50. The assay kit of the forty-ninth embodiment, further comprising structural elements for:

(a) immobilizing HERV protein onto a solid surface;

(b) adding a bodily fluid sample suspected of containing anti-HERV antibodies or autoantibodies, obtained from a subject suspected of suffering from cancer, to said immobilized HERV protein so as to allow a complex formation between HERV protein and said anti-HERV antibodies or autoantibodies, wherein said anti-HERV antibodies or autoantibodies recognize the immobilized HERV protein; and

(c) detecting said protein-antibody complex, wherein the presence of said protein-antibody complex is indicative of a cancer in said subject.

51. The method of the thirty-ninth embodiment, further comprising the steps of:

(b) immobilizing a first anti-HERV antibody onto a solid surface;

(b) adding a bodily fluid sample suspected of containing HERV protein, obtained from a subject suspected of suffering from cancer, onto said solid surface having said immobilized first anti-HERV antibody and allowing formation of HERV protein and first anti-HERV antibody complex;

(d) removing unbound HERV protein;

(e) adding a second anti-HERV antibody so as to allow formation of a complex between said bound HERV protein with said second anti-HERV antibody; and

(f) detecting said bound HERV protein with said second anti-HERV antibody complex, wherein said first anti-HERV antibody and said second anti-HERV antibody recognize a different region of HERV protein, and wherein the presence of said protein-antibody complex is indicative of a cancer in said subject.

52. The method of the thirty-ninth embodiment, further comprising a kit including:

(a) a container for a bodily fluid sample;

(b) a microtiter plate;

(c) a detection reagent;

(d) a first anti-HERV antibody, said first anti-HERV antibody recognizes HERV protein;

(e) a second anti-HERV antibody, said second anti-HERV-antibody also recognizes HERV, said second anti-HERV antibody having a recognition site different from that of said first anti-HERV antibody; and

(f) an instruction for the use of said antibody in detecting HERV protein in an ELISA.

15. The method of the thirty-ninth embodiment, further comprising the steps of:

(a) obtaining a biological sample from a subject suspected of suffering from cancer; and

(b) detecting HERV in said biological sample using an anti-HERV antibody in an immunoblot assay, wherein the presence of HERV protein in said biological sample is indicative of a cancer in the subject suspected of suffering from cancer.

53. The method of the thirty-ninth embodiment, further comprising the steps of:

(a) obtaining a biological sample from a subject suspected of suffering from cancer; and

(b) detecting HERV in said biological sample using an anti-HERV antibody in an immunohistochemistry assay, wherein the presence of HERV protein in said biological sample is indicative of a cancer in the subject suspected of suffering from cancer.

54. The method of the thirty-ninth embodiment, further comprising the steps of:

(a) obtaining a biological sample from a subject suspected of suffering from cancer; and

(b) detecting HERV in said biological sample using polymerase chain reaction (PCR), reverse transcriptase PCR (RT-PCR), or quantitative real-time RT-PCR (qRT-PCR) assays, wherein the presence of HERV RNA in said biological sample is indicative of a cancer in the subject suspected of suffering from cancer.

55. The method of the thirty-ninth embodiment, further comprising the steps of:

(a) obtaining a biological sample from a subject suspected of suffering from cancer; and

(b) detecting reverse transcriptase enzyme activity in said biological sample using a reverse transcriptase enzyme activity assay, wherein the presence of reverse transcriptase enzyme activity in said biological sample is indicative of a cancer in the subject suspected of suffering from cancer.

56. The method of the thirty-ninth embodiment, further comprising the steps of:

(a) labeling anti-HERV antibodies with 99Tc or labeling with another gamma-ray emitting isotope;

(b) imaging HERV distribution in tumors in vivo with a gamma scintillation camera and assess stained tissues for radioactivity counting as an indicator of the amount of HERV-associated peptides in the tumor as a biomarker for the presence of invasive cancer cells;

(c) using in vivo magnetic resonance imaging (MRI) of HERV to improve imaging over gamma camera techniques;

(d) using in vivo imaging of HERV to allow for the identification and localization of a tumors in a cancer patient;

(e) using in vivo imaging of HERV to detect micrometastases in a human patient

(f) using in vivo imaging with monoclonal anti-HERV antibodies to indicate the presence of aggressive or invasive carcinomas;

(g) using in vivo imaging of HERV to guide therapy with anti-HERV therapeutic antibodies;

(h) using anti-HERV antibody conjugates with dyes (biotin-streptavidin complex), contrast agents (such as for magnetic resonance imaging, computed tomography, or ultrasound contrast-enhancing agent), fluorescent compounds or molecules and enhancing agents (e.g. paramagnetic ions) for magnetic resonance imaging (MRI) to improve sensitivity of detection by in vivo imaging.

57. The assay kit of the forty-ninth embodiment, wherein the test device for detecting, diagnosing and following progression of cancer in a human subject is a lateral flow immunoassay.

58. The assay kit of the fifty-seventh embodiment, wherein said lateral flow immunoassay comprises, for each molecule to be assayed, a capture agent selected from the group consisting of an antibody, a portion of an antibody, a single chain antibody, a non-immunoglobulin receptor for the molecule, a peptide ligand for the molecule, and an oligonucleotide ligand for the molecule.

59. The assay kit of the fifty-seventh embodiment, wherein said capture agent is bound to a solid support.

60. A method of detecting cancer in a patient, comprising:

(i) obtaining a biological sample from a subject suspected of suffering from cancer, and

(ii) identifying, in the biological sample, an increase in the levels of at least one of the biomarker molecules described in the thirty-ninth embodiment relative to a healthy control subject.

61. The method of the sixtieth embodiment, wherein the HERV is HERV-K.

62. The method of the sixtieth embodiment, wherein the biological sample is a bodily fluid.

63. The method of the sixty-second, wherein the bodily fluid comprises peripheral blood, sera, plasma, ascites, urine, cerebrospinal fluid (CSF), sputum, saliva, bone marrow, synovial fluid, aqueous humor, cerumen, bronchioalveolar lavage fluid, semen, prostatic fluid, Cowper's fluid or pre-ejaculatory fluid, sweat, fecal matter, tears, cyst fluid, pleural fluid, peritoneal fluid, pericardial fluid, lymph, chyme, chyle, bile, interstitial fluid, pus, sebum, vomit, mucosal secretion, stool water, pancreatic juice, lavage fluids from sinus cavities, bronchopulmonary aspirates, blastocyl cavity fluid, or a combination thereof.

64. The method of the sixtieth embodiment, further comprising determining whether the levels of at least one of the molecules are higher than a control level, wherein the control level is determined from a group of healthy subjects or subjects without cancer.

65. The method of the sixtieth embodiment, further comprising determining whether an increase in the levels of at least one of the molecules, relative to a healthy subject or subject without cancer, indicates the presence of cancer in the subject.

66. The method of the sixtieth embodiment, further comprising selecting a therapy for cancer if the levels of at least one of the molecules are higher than the levels in a healthy subject or subject without cancer.

67. The method of the sixtieth embodiment, comprising selecting a subject identified as having cancer for confirmatory diagnostic cancer testing.

68. The method of the sixtieth embodiment, further comprising administering a therapy for cancer if the levels of at least one of the molecules are higher than the levels in a healthy subject or subject without cancer.

69. An assay kit for detecting HERV and HERV-associated-molecule biomarkers for cancer in a patient consisting of

(a) antigens or antibodies immunologically specific for at least one of the HERV and HERV-associated-molecule biomarkers described in the thirty-ninth embodiment for detecting expression levels of said HERV and HERV-associated-molecule biomarkers in a sample obtained from said patient, wherein said labeled antigens or antibodies form specific binding pairs with said HERV and HERV-associated-molecule biomarkers, and wherein said labeled HERV and HERV-associated-molecule biomarkers are suitable for flow cytometric analysis, immunohistochemical detection, or immunoblot analysis, or labeled nucleic acids or nucleic acid primers which specifically hybridize to or amplify HERV and HERV-associated-molecule biomarkers for detecting expression levels of said HERV and HERV-associated-molecule biomarkers in a sample obtained from said patient, wherein said labeled nucleic acids amplify said HERV and HERV-associated-molecule biomarker-encoding nucleic acids, and wherein said labeled nucleic acids are suitable for performance of in situ hybridization assay, hybridization assay, gel electrophoresis, RT-PCR, real time PCR, or microarray analysis, and

(b) optionally, instructional materials comprising ranges of HERV and HERV-associated-molecule biomarker expression levels associated with aggressive metastatic cancer and ranges of expression levels associated with non-aggressive non-metastatic cancer.

70. An assay kit for detecting, diagnosing, and following progression of cancer in a subject, comprising one or more reagents sufficient for detection of the presence or absence of an anti-HERV antibody or an HERV target in a sample, for determining the activity of cancer cells toward agents selectively targeting HERV. The kit consists of a preparation of cancer cells; one or more agents selectively targeting HERV; and one or more reagents sufficient to perform an assay selected from groups that comprise (1) cell growth or survival assays carried out under specific culture conditions, (2) the ability to express a defined biologic factor, (3) cell structure assays, or (4) differential gene expression assays. Kit components include primers, buffers, probes, antibodies (primary and secondary) for immunolabeling and signal detection to increase signal amplification and sensitivity. Secondary antibodies can be conjugated to enzymes such as horseradish peroxidase (HRP) or alkaline phosphatase (AP); or fluorescent dyes such as fluorescein isothiocyanate (FITC), rhodamine derivatives, Alexa Fluor dyes, or other molecules to be used in various applications, enzymes, e.g., polymerases, ligases, reverse transcriptase's, nucleases, etc., components for sample isolation, sample preparation, instrumentation, software, instructions for cancer detection, diagnosis, or prognosis in the subject based on the presence or absence of targets that comprise HERV antigens, HERV nucleic acids, or anti-HERV antibodies described in the thirty-ninth embodiment. The instructions optionally provide recommendations to assist a treating physician in the course of action, based on the results of the analysis, to optimize patient care.

71. The method of the sixtieth embodiment, further comprising the steps of:

(a) immobilizing HERV protein onto a solid surface;

(b) adding a bodily fluid sample suspected of containing anti-HERV antibodies or autoantibodies, obtained from a subject suspected of suffering from cancer, to said immobilized HERV protein so as to allow a complex formation between HERV protein and said anti-HERV antibodies or autoantibodies, wherein said anti-HERV antibodies or autoantibodies recognize the immobilized HERV protein; and

(c) detecting said protein-antibody complex, wherein the presence of said protein-antibody complex is indicative of a cancer in said subject.

72. The method of the sixtieth embodiment, further comprising the steps of:

(a) immobilizing a first anti-HERV antibody onto a solid surface;

(b) adding a bodily fluid sample suspected of containing HERV protein, obtained from a subject suspected of suffering from cancer, onto said solid surface having said immobilized first anti-HERV antibody and allowing formation of HERV protein and first anti-HERV antibody complex;

(c) removing unbound HERV protein;

(d) adding a second anti-HERV antibody so as to allow formation of a complex between said bound HERV protein with said second anti-HERV antibody; and

(e) detecting said bound HERV protein with said second anti-HERV antibody complex, wherein said first anti-HERV antibody and said second anti-HERV antibody recognize a different region of HERV protein, and wherein the presence of said protein-antibody complex is indicative of a cancer in said subject.

73. An assay kit including:

(a) a container for a bodily fluid sample;

(b) a microtiter plate;

(c) a detection reagent;

(d) a first anti-HERV antibody, said first anti-HERV antibody recognizes HERV protein;

(e) a second anti-HERV antibody, said second anti-HERV-antibody also recognizes HERV, said second anti-HERV antibody having a recognition site different from that of said first anti-HERV antibody; and

(f) an instruction for the use of said antibody in detecting HERV protein in an ELISA.

74. The method of the sixtieth embodiment, further comprising the steps of:

(a) obtaining a biological sample from a subject suspected of suffering from cancer; and

(b) detecting HERV in said biological sample using an anti-HERV antibody in an immunoblot assay, wherein the presence of HERV protein in said biological sample is indicative of a cancer in the subject suspected of suffering from cancer.

75. The method of the sixtieth embodiment, further comprising the steps of:

(a) obtaining a biological sample from a subject suspected of suffering from cancer; and

(b) detecting HERV in said biological sample using an anti-HERV antibody in an immunohistochemistry assay, wherein the presence of HERV protein in said biological sample is indicative of a cancer in the subject suspected of suffering from cancer.

76. The method of the sixtieth embodiment, further comprising the steps of:

(a) obtaining a biological sample from a subject suspected of suffering from cancer; and

(b) detecting HERV in said biological sample using polymerase chain reaction (PCR), reverse transcriptase PCR (RT-PCR), or quantitative real-time RT-PCR (qRT-PCR) assays,

wherein the presence of HERV RNA in said biological sample is indicative of a cancer in the subject suspected of suffering from cancer.

77. The method of the sixtieth embodiment, further comprising the steps of:

(a) obtaining a biological sample from a subject suspected of suffering from cancer; and

(b) detecting reverse transcriptase enzyme activity in said biological sample using a reverse transcriptase enzyme activity assay, wherein the presence of reverse transcriptase enzyme activity in said biological sample is indicative of a cancer in the subject suspected of suffering from cancer.

78. The method of the sixtieth embodiment, further comprising the steps of:

(a) labeling anti-HERV antibodies with 99Tc or labeling with another gamma-ray emitting isotope;

(b) imaging HERV distribution in tumors in vivo with a gamma scintillation camera and assess stained tissues for radioactivity counting as an indicator of the amount of HERV-associated peptides in the tumor as a biomarker for the presence of invasive cancer cells;

(c) using in vivo magnetic resonance imaging (MRI) of HERV to improve imaging over gamma camera techniques;

(d) using in vivo imaging of HERV to allow for the identification and localization of a tumors in a cancer patient;

(e) using in vivo imaging of HERV to detect micrometastases in a human patient;

(f) using in vivo imaging with monoclonal anti-HERV antibodies to indicate the presence of aggressive or invasive carcinomas;

(g) using in vivo imaging of HERV to guide therapy with anti-HERV therapeutic antibodies;

(h) using anti-HERV antibody conjugates with dyes (biotin-streptavidin complex), contrast agents (such as for magnetic resonance imaging, computed tomography, or ultrasound contrast-enhancing agent), fluorescent compounds or molecules and enhancing agents (e.g. paramagnetic ions) for magnetic resonance imaging (MRI) to improve sensitivity of detection by in vivo imaging.

79. An assay device for detecting, diagnosing and following progression of cancer in a human subject, wherein the device is a lateral flow immunoassay.

80. The device of the seventy-ninth embodiment, wherein said lateral flow immunoassay comprises, for each molecule to be assayed, a capture agent selected from the group consisting of an antibody, a portion of an antibody, a single chain antibody, a non-immunoglobulin receptor for the molecule, a peptide ligand for the molecule, and an oligonucleotide ligand for the molecule.

81. The device of the seventy-ninth embodiment, wherein said capture agent is bound to a solid support.

82. An assay kit for detecting HERV-associated cancer biomarkers for use in detecting cancer in a subject, comprising:

(a) a reagent capable of specifically binding to HERV antigens, HERV-associated antibodies, or HERV targets, wherein the HERV targets are HERV polynucleotides;

(b) a means for detecting the specific binding of the reagent to the HERV antigens, HERV-associated antibodies, or HERV targets.

83. A biomarker panel for use in detecting cancer in a patient, comprising one or more of the following markers detected in a biological sample from a subject:

(a) the LTR, gag, pol, or env regions of HERV-K, optionally wherein the HERV-K variant is Np9/Rec;

(b) the LTR, gag, pol, or env regions of HERV-H, HERV-H5, HERV-H-48-1;

(c) the LTR, gag, pol, or env regions of HERV-E;

(d) the LTR, gag, pol, or env regions of HERV3

(e) LTR, gag, pol, or env regions of HERV-E, HERV-W, or HERV-R, I, T, P, L;

(f) an immune checkpoint inhibitor selected from the group CD27; LAG-3; TIM-3; CTLA-4; PD-L1; PD-L2; BTLA; CD28; IDO; CD80; 4-1BB; HVEM; or GITR.

The phylogenetic analysis shows three main branches of HERVs. The first main branch was made up of HERV-H, HER-F, HERV-S71-related, ERV9, MSRV, HERV-K1.1, HERV-E, HERV-R, HERV-I, RTLVH, HERV-S and HRES-1. The second main branch was composed of HERV-T, HERV-P, HERV-FRD, HERV-KHTDV, and HERV-W. The third main branch predominately contains the HERV-K family, HERV-L, HERV-PT47D and XMRV. The HERV-Ks family is the most homologous among all the HERVs and also ubiquitous in terms of cancer tissue expression. The youngest sub-class of the HERV-Ks-HERV-K 133, and HERV-KHML 1.1, together with the widely debated XMRV are nested in the same group and present about 60% similarity. HERV-F and HERV-H present 70% similarity. HERV-S-71-Related, ERV9 and MSRV showed 100% similarity.

### REFERENCES

A person having ordinary skill in the art can use these patents, patent applications, and scientific references as guidance to predictable results when making and using the invention.

### Patent Literature

U.S. Pat. No. 5,902,725 (Robbins et al.) describes methods for detecting cancer specific antigens, e.g., detection of prostate specific antigen (PSA).
U.S. Pat. No. 9,243,055 (Wang-Johanning) discloses methods and compositions for cancer diagnostics and therapy, and methods and compositions for detecting, preventing, and treating HERV-K+ cancers. The method may involve a method for preventing or inhibiting cancer cell proliferation by administering to a subject a cancer cell proliferation blocking or reducing amount of a HERV-K env protein binding antibody.

### Non-Patent Literature

Buscher et al., Expression of human endogenous retrovirus K in melanomas and melanoma cell lines. Cancer Research, 65(10), 4172-80 (2005).
Chae et al., Epithelial-mesenchymal transition (EMT) signature is inversely associated with T-cell infiltration in non-small cell lung cancer (NSCLC). Science Reports, 8(1), 2918 (2018).
Denne et al., Physical and functional interactions of human endogenous retrovirus proteins Np9 and rec with the promyelocytic leukemia zinc finger protein. J. Virol., 81(11), 5607-16 (2007).
Downey et al., Human endogenous retrovirus K and cancer: Innocent bystander or tumorigenic accomplice? Internal Journal of Cancer, 137(6), 1249-57 (2015).
Ejthadi et al., A novel multiplex RT-PCR system detects human endogenous retrovirus-K in breast cancer. Arch. Virol., 150(1), 177-84 (2005).
Etkind et al., Type 1 HERV-K genome is spliced into subgenomic transcripts in the human breast tumor cell line T47D. Virology 1997;234(2), 304-8.
Fischer et al., Human endogenous retrovirus np9 gene is over expressed in chronic lymphocytic leukemia patients. Leuk. Res. Rep., 3(2), 70-2 (2014).
Gonzalez-Cao et al., Human endogenous retroviruses and cancer. Cancer Biol. Med., 13(4), 483-88 (2016).
Haraguchi, Good, & Day-Good, A potent immunosuppressive retroviral peptide: cytokine patterns and signaling pathways. Immunol. Res., 41(1), 46-55 (2008).
Hughes & Coffin J M. Evidence for genomic rearrangements mediated by human endogenous retroviruses during primate evolution. Nature Genetics, 29(4), 487-9 (2001).
Hummel et al., Human endogenous retrovirus envelope proteins target dendritic cells to suppress T-cell activation. Eur. J. Immunol., 45(6), 1748-59 (2015).
Johanning et al., Expression of human endogenous retrovirus-K is strongly associated with the basal-like breast cancer phenotype. Science Reports, 7, 41960 (2017).
Kleiman et al. HERV-K(HML-2) GAG/ENV antibodies as indicator for therapy effect in patients with germ cell tumors. Internal Journal of Cancer, 110(3), 459-61 (2004).
Kraus et al., Vaccination directed against the human endogenous retrovirus-K envelope protein inhibits tumor growth in a murine model system. PLoS One, 8(8), e72756 (2013).
Kudo-Saito et al., Induction of immunoregulatory CD271+ cells by metastatic tumor cells that express human endogenous retrovirus H. Cancer Research, 74(5), 1361-70 (2014).
Lander et al., Initial sequencing and analysis of the human genome. Nature, 409(6822), 860-921 (2001).
Larsson Kato, & Cohen, Human endogenous proviruses. Current Topics Microbiol. Immunol., 148, 115-32 (1989).
Lemaitre et al., A human endogenous retrovirus-derived gene that can contribute to oncogenesis by activating the ERK pathway and inducing migration and invasion. PLoS Pathog., 13(6), e1006451 (2017).
Li et al. Down-regulation of human endogenous retrovirus type K (HERV-K) viral env RNA in pancreatic cancer cells decreases cell proliferation and tumor growth. Clinical Cancer Research (2017).
Li et al., Downregulation of Human Endogenous Retrovirus Type K (HERV-K) Viral env RNA in Pancreatic Cancer Cells Decreases Cell Proliferation and Tumor Growth. Clinical Cancer Research, 23(19), 5892-911 (2017).
Li et al., Down-regulation of human endogenous retrovirus type K (HERV-K) viral env RNA in pancreatic cancer cells decreases cell proliferation and tumor growth. Clinical Cancer Research, 23(19), 5892-5911 (Oct. 1, 2017).
Morozov, Dao Thi, & Denner, The transmembrane protein of the human endogenous retrovirus-K (HERV-K) modulates cytokine release and gene expression. PLoS One, 8(8), e70399 (2013).
Ono et al., Nucleotide sequence of human endogenous retrovirus genome related to the mouse mammary tumor virus genome. J. Virol., 60(2), 589-98 (1986).
Ono, Kawakami, & Ushikubo, Stimulation of expression of the human endogenous retrovirus genome by female steroid hormones in human breast cancer cell line T47D. J. Virol., 61(6), 2059-62 (1987).
Oricchio et al., Distinct roles for LINE-1 and HERV-K retroelements in cell proliferation, differentiation and tumor progression. Oncogene, 26(29), 4226-33 (2007).
Pitot et al., Fundamentals of Oncology (Marcel Dekker Inc., 1978)), pp. 15-28.
Rycaj et al., Cytotoxicity of human endogenous retrovirus K-specific T cells toward autologous ovarian cancer cells. Clinical Cancer Research, 21(2), 471-83 (2015).
Seifarth et al., Retrovirus-like particles released from the human breast cancer cell line T47-D display type B- and C-related endogenous retroviral sequences. J. Virol., 69(10), 6408-16 (1995)
Serafino et al., The activation of human endogenous retrovirus K (HERV-K) is implicated in melanoma cell malignant transformation. Exp. Cell Res. 315(5), 849-62 (2009).
Siegel, Miller, & Jemal, Cancer statistics, 2018. CA Cancer J. Clin., 68(1), 7-30 (2018).
Wallace et al., Elevated HERV-K mRNA expression in PBMC is associated with a prostate cancer diagnosis particularly in older men and smokers. Carcinogenesis, 35(9), 2074-83 (2014).
Wang-Johanning et al. Immunotherapeutic potential of anti-human endogenous retrovirus-K envelope protein antibodies in targeting breast tumors. J. Natl. Cancer Inst., 104(3), 189-210 (2012).
Wang-Johanning et al. Human endogenous retrovirus K triggers an antigen-specific immune response in breast cancer patients. Cancer Research, 68(14), 5869-77 (2008).
Wang-Johanning et al. Expression of human endogenous retrovirus k envelope transcripts in human breast cancer. Clinical Cancer Research, 7(6), 1553-60 (2001).
Wang-Johanning et al. Expression of multiple human endogenous retrovirus surface envelope proteins in ovarian cancer. Internal Journal of Cancer, 120(1), 81-90.
Wang-Johanning et al., Detecting the expression of human endogenous retrovirus E envelope transcripts in human prostate adenocarcinoma. Cancer, 98(1), 187-97 (2003).
Wang-Johanning et al., Quantitation of HERV-K env gene expression and splicing in human breast cancer. Oncogene, 22(10), 1528-35 (2003).
Wang-Johanning et al., Human endogenous retrovirus type K antibodies and mRNA as serum biomarkers of early-stage breast cancer. Internal Journal of Cancer, 134(3), 587-95 (2014).
Wang-Johanning et al., Tumor microenvironment predicts aggressive breast cancer: Combination of HERV-K, immune checkpoint and activation status of CD8+ T cells. Cancer Research, 77(13 Suppl), Abstract nr LB-221 (2017).
Zhao et al., Expression of Human Endogenous Retrovirus Type K Envelope Protein is a Novel Candidate Prognostic Marker for Human Breast Cancer. Genes Cancer, 2(9), 914-22 (2011).
Zhou et al., Activation of HERV-K Env protein is essential for tumorigenesis and metastasis of breast cancer cells. Oncotarget, 7(51), 84093-117 (2016).
Zhou et al., Chimeric antigen receptor T cells targeting HERV-K inhibit breast cancer and its metastasis through downregulation of Ras. Oncoimmunology, 4(11), e1047582 (2015).

We claim:

1. An assay kit for detecting HERV-associated cancer biomarkers for use in detecting cancer in a subject, comprising:
(a) a reagent capable of specifically binding to HERV antigens, HERV-associated antibodies, HERV targets, wherein the HERV targets are HERV polynucleotides, or a marker of HERV expression in the subject;
(b) a means for detecting the specific binding of the reagent to the HERV antigens, HERV-associated antibodies, or HERV targets.

2. The assay kit of claim 1, wherein reagent capable of specifically binding to a HERV antigen is an anti-HERV antibody

3. The assay kit of claim 1, wherein the reagent capable of specifically binding to a HERV antigens is an anti-HERV antibody conjugated to a detection agent or contrast agent.

4. The assay kit of claim 1, wherein the HERV-associated antibodies are the subject's autoantibodies.

5. The assay kit of claim 1, wherein the HERV-associated antibodies are the subject's autoantibodies.

6. The assay kit of claim 1, wherein the HERV target is detected by RT-PCR.

7. The assay kit of claim 1, wherein the marker of HERV expression in the subject is reverse transcriptase.

8. The assay kit of claim 1, wherein the means for detecting the specific binding of the reagent increases the signal amplification and sensitivity.

9. The assay kit of claim 1, wherein the reagent capable of specifically binding to HERV antigens comprises secondary antibodies conjugated to enzymes such as horseradish peroxidase (HRP) or alkaline phosphatase (AP); or fluorescent dyes such as fluorescein isothiocyanate (FITC), rhodamine derivatives, or Alexa Fluor dyes.

11. The assay kit of claim 1, further comprising instructions that provide recommendations to assist a treating physician in the course of action, based on the results of the analysis, to optimize patient care.

12. A biomarker panel for use in detecting cancer in a patient, comprising one or more of the following markers detected in a biological sample from a subject:
(a) the LTR, gag, pol, or env regions of HERV-K, optionally wherein the HERV-K variant is Np9/Rec;
(b) the LTR, gag, pol, or env regions of HERV-H, HERV-H5, or HERV-H48-1;
(c) the LTR, gag, pol, or env regions of HERV-E;
(d) the LTR, gag, pol, or env regions of HERV3
(e) the LTR, gag, pol, or env regions of HERV-E, HERV-W, or HERV-R, I, T, P, L;
(f) an immune checkpoint inhibitor selected from the group CD27; LAG-3; TIM-3; CTLA-4; PD-L1; PD-L2; BTLA; CD28; IDO; CD80; 4-1BB; HVEM; or GITR.

13. A method of detecting cancer in a subject, comprising the steps of:
(i) obtaining a biological sample from the subject, and
(ii) identifying, in the biological sample, an increase in the levels of at least one of the following molecules relative to a healthy control subject: anti-human endogenous retrovirus (HERV) protein antibody; anti-HERV peptide antibody; anti-HERV Env surface (SU) protein antibody; anti-HERV Env surface (SU) peptide antibody; anti-HERV Env transmembrane (TM) protein antibody; anti-HERV Env transmembrane (TM) peptide antibody; anti-HERV Gag protein antibody; anti-HERV Gag peptide antibody; anti-HERV Pol protein antibody; anti-HERV Pol peptide antibody; anti-HERV NP9 protein antibody; anti-HERV NP9 peptide antibody; anti-HERV Rec protein antibody; anti-HERV Rec peptide antibody; anti-HERV multiple antigen peptide (MAP) antibody; HERV protein; HERV peptide; HERV Env surface (SU) protein; HERV Env surface (SU) peptide; HERV Env transmembrane (TM) protein; HERV Env transmembrane (TM) peptide; HERV Gag protein; HERV Gag peptide; HERV Pol protein; HERV Pol peptide; HERV NP9 protein; HERV NP9 peptide; HERV Rec protein; HERV Rec peptide; HERV RNA; HERV Env surface (SU) RNA; HERV Env transmembrane (TM) RNA; HERV Gag RNA; HERV Pol RNA HERV NP9 RNA; HERV Rec RNA; HERV DNA; HERV Env surface (SU) DNA; HERV Env transmembrane (TM) DNA; HERV Gag DNA; HERV Pol DNA; HERV NP9 DNA; HERV Rec DNA; immune checkpoint molecule PD-1 protein; immune checkpoint molecule PD-1 RNA; immune checkpoint molecule PD-L1 protein; immune checkpoint molecule PD-L1 RNA; immune checkpoint molecule PD-L2 protein; immune checkpoint molecule PD-L2 RNA; immune checkpoint molecule CTLA-4 protein; immune checkpoint molecule CTLA-4 RNA; immune checkpoint molecule LAG3 protein; immune checkpoint molecule LAG3 RNA; immune checkpoint molecule TIM3 protein; immune checkpoint molecule TIM3 RNA; immune checkpoint molecule CD27 protein; immune checkpoint molecule CD27 RNA; immune checkpoint molecule B7 family protein; immune checkpoint molecule B7 family RNA; HERV reverse transcriptase (RT) activity; a control (consensus) HERV immunosuppressive domain region (TMc) protein sequence; a control (consensus) HERV immunosuppressive domain region (TMc) RNA sequence; a control (consensus) HERV immunosuppressive domain region (TMc) protein with sequence GIHWQKNSARLWNSQSSIDQKLANQINDLRQTVIWMGDRLMSLEHRFQ LQCDC (SEQ ID NO: 1); a HERV RNA sequence that codes for a control (consensus) (TMc) immunosuppressive domain region with protein sequence GIHWQKNSARLWNSQSSIDQKLANQINDLRQTVIWMGDRLMSLEHRFQ LQCDC (SEQ ID NO: 1); a variant HERV immunosuppressive domain region (TMv) region protein with sequence; IGKRILQDCGIHNLVLIKNWQIKLIILDKLIWIGDRLMSLERRFQLQCDC (SEQ ID NO: 2); and an RNA sequence that codes for a variant HERV immunosuppressive domain region (TMv) region with protein sequence IGKRILQDCGIHNLVLIKNWQIKLIILDKLIWIGDRLMSLERRFQLQCDC (SEQ ID NO: 2); and
(iii) selecting a cancer therapy for the subject if the levels of at least one of the molecules are higher than the levels in a healthy subject or subject without cancer.
